SEQUENCE LISTING (272 sequences)

SEQ ID NO: 1 - protein, 26 aa, Camelus sp.
QVQLQESGGGLVQAGGSLRLSCAASG

SEQ ID NO: 2 - protein, 14 aa, Camelus sp.
WFRQAPGKEREFVA

SEQ ID NO: 3 - protein, 33 aa, Camelus sp.
GRFTISRDNAKNTVYLQMNSLKPEDTAVYYCAA

SEQ ID NO: 4 - protein, 11 aa, Camelus sp.
WGQGTQVTVSS

SEQ ID NO: 5 - protein, 126 aa, Camelus sp.
QVQLQESGGGLVQAGGSLRLSCAASGFTFDDYAIGWFRQAPGKEREGVSCLKSSDGRIYYADSVKGRFTISSDNAKNTVYLQMNSLKPEDTAVYYCAAERKFYGLGYCSEYGMDYWGKGTLVTVSS

SEQ ID NO: 6 - protein, 127 aa, Camelus sp.
QVQLQESGGGLVQAGGSLRLSCAASGRTFSSYAMGWFRQAPGKEREFVAGISGSGGRTYYEDSVKGRFTITRDNGKSVVYLQMNSLELEDTAVYYCAANLRGYNSGTYYRASDFDSWGQGTQVTVSS

SEQ ID NO: 7 - protein, 126 aa, Camelus sp.
QVQLQESGGGLVQAGGSLRLSCAASGRTFSSYAMGWFRQAPGKEREFVAAISWSGDSTNYADSVKGRFTISRDNAKNTVYLQMNSLKPEDTAVYYCAADSHYGYSEYIRGLGMDYWGKGTLVTVSS

SEQ ID NO: 8 - protein, 127 aa, Camelus sp.
QVQLQDSGGGLVQAGGSLRLSCAASERTFSTYAMGWFRQAPGKEREFVAGISGSGGRTYYEDSVKGRFTISRDNAKSMVYLQMNSLKLEDTAVYYCAAKPRGYNSGTYYRASDFDSWGQGTQVTVSS

SEQ ID NO: 9 - protein, 126 aa, Camelus sp.
QVQLQDSGGGLVQAGGSLRLSCAASGRTFSSYAMGWFRQAPGKEREFVAAISFSGDSTAYADSVKGRFTISRDNAKNTVYLQMNSLKPEDTAVYYCAADSHYGYSEYIRGLGMDYWGKGTLVTVSS

SEQ ID NO: 10 - protein, 124 aa, Camelus sp.
QVQLQESGGGLVQTGDSLRLSCAVSGRTVNSYAMAWFRQAPGKEREFVAATTWRGTSTNYADPVKGRFTMSRDNAKNTVDLQMNSLKPEDTAVYYCAANTYYRGGGYLTEYNLWGQGTQVTVSS

SEQ ID NO: 11 - protein, 127 aa, Camelus sp.
QVQLQESGGGLVQAGGSLRLSCAASERTFSTYAMGWFRQAPGKEREFVAGISGSGGRTYYEDSVKGRFTISRDNAKSMVYLQMNSLKLEDTAVYYCAAKPRGYNSVTYYRASDFDSWGQGTQVTVSS

SEQ ID NO: 12 - protein, 126 aa, Camelus sp.
QVQLQESGGGLVQAGGSLRLSCLASGRTFSSYAMGWFRQAPGKEREFLATINWSGGTTYYADSVKGRFTVSRDNAKNTVYLQMNSLKPEDTAVYYCAAVRYGGGGFFTDSKSFDYWGQGTQVTVSS

SEQ ID NO: 13 - protein, 126 aa, Camelus sp.
QVQLQESGGGLVQAGGSLRLSCAASGRTFSSYAMGWFRQAPGKEREFVAAISFSGDSTDYADSVKGRFTISRDNAKNTVYLQMNSLKPEDTAVYYCAADSHYGYSEYIRGLGMDYWGKGTLVTVSS

SEQ ID NO: 14 - protein, 127 aa, Camelus sp.
QVQLQESGGGLVQAGGSLRLSCAASERTFSTYAMGWFRQAPGKEREFVAGISGSGGRTYYEDSVKGRFTISRDNAKSMVYLQMNSLKLEDTAVYYCAAKPRGYNSGTYYRASDFDSWGQGTQVTVSS

SEQ ID NO: 15 - protein, 124 aa, Camelus sp.
QVQLQDSGGGLVQPGGSLRLSCAASGLSLESTVVGWFRQAPGKEREGISCISSGDTNTLYVDSVKGRFTISRDNAKNTVYLDMNRLKPEDTAVYYCATGVDSGYGCPAYEYDSWGQGTQVTVSS

SEQ ID NO: 16 - protein, 126 aa, Camelus sp.
QVQLQESGGGLVQAGDSLRLSCAASGRTFSSYAMAWFRQAPGKDREFVGTINWSGSSTYYVDSVKGRFTISRDNAKNTVYLQMNSLKPEDTAVYYCAASHYGGSGYLLEQRSYAYWGQGTQVTVSS

SEQ ID NO: 17 - protein, 126 aa, Camelus sp.
QVQLQESGGGLVQAGDSLRLSCAASGRTFSAYTVGWFRQAPGKKREFVATVNWSGGSSYYADSVKGRFTISRDNAKNTVYLQMNSLKPEDTAVYSCAATEYGGSGYLLGGLDYHYWGQGTQVTVSS

SEQ ID NO: 18 - protein, 127 aa, Camelus sp.
QVQLQESGGGLVQAGGSLRLSCAASGRTFSSYAMGWFRQAPGKEREFVAGISGSGGRTYYEDSVKGRFTISRDNAKNMVYLQMNSLKLEDTAVYYCAANTRGYNSGTYYRASDFDSWGQGTQVTVSS

SEQ ID NO: 19 - protein, 126 aa, Camelus sp.
QVQLQDSGGGLVQAGGSLRLSCAASGPTFSRYAMGWFRQAPGKEREFVAAISWSGDRTDYAESVKGRFTISRDNAKNTVYLQMNSLKPEDTAVYYCAADSHYGYSEYIRGLGMDYWGKGTLVTVSS

SEQ ID NO: 20 - protein, 126 aa, Camelus sp.
QVQLQESGGGSVRAGDSLRLSCAASGRTVSTSAMAWFRQAPGKERQFVASISWTGDSTNYADSVRGRFTISKEATENTNYLQMNVLKPEDTAVYYCAAGSGIYAYLYTDHRAYEYWGQGTQVSVSS

SEQ ID NO: 21 - protein, 128 aa, Camelus sp.
QVQLQESGGGLVQAGGSLRLSCAASERTFSTYAMGWFRQAPGKEREFVAGISGSGGRTYYEDSVKGRFTISRDNAKSMVYLQMNSLKLEDTAVYYCAAKPRGYNSGTYYRASDFDFPGGQGTQVTVSS

SEQ ID NO: 22 - protein, 127 aa, Camelus sp.
QVQLQESGGGLVQAGGSLRLSCAASGRTFSSYAMGWFRQAPGKEREFVAGISGSGGRTYYEDSVKGRFTISRDNAKNMVYLQMISLKLEDTAVYYCAANTRGYNSGSYYRASDFDSWGQGTQVTVSS

SEQ ID NO: 23 - protein, 129 aa, Camelus sp.
QVQLQDSGGGLVQAGGSLRLSCAASGFTGYTFDDYAIGWFRQAPGKEREGVSCISSSDGTTYYADSVKGRFTISSDNAKNTVTLQMNGLKPEDTAVYYCAADLFTSYCSDPMHPRYDYWGQGTQVTVSS

SEQ ID NO: 24 - protein, 128 aa, Camelus sp.
QVQLQDSGGGLVQAGESLRLSCAASERTFSTYAMGWFRQAPGKEREFVAGISGSGGRTYYEDSVKGRFTISRDNAKSMVYLQMNSLKLEDTAVYYCAAANPRGYNSGTYYRASDFDSWGQGTQVTVSS

SEQ ID NO: 25 - protein, 126 aa, Camelus sp.
QVQLQDSGGGLVQAGGSLRLSCAASGRTYSSYAMGWFRQAPGKEREFVAAINWSGGTTYYTDSVKGRFTISRDNAKNTVYLQMNSLKSEDTAVYYCAATYYGGGGFFTESHQYDYWGQGTQVTVSS

SEQ ID NO: 26 - protein, 127 aa, Camelus sp.
QVQLQDSGGGLVQAGGSLRLSCAASGFTFNDYAIGWFRQAPGKEREGVSWISSSDGSTKYADSVKGRFTISSDNAKNTVYLQMNSLQPEDTAVYYCAAAQLFAGSYYYTAPEWYDYWGQGTRVTVSS

SEQ ID NO: 27 - protein, 126 aa, Camelus sp.
QVQLQESGGGLVQAGGSLRLSCAASGLYVSANDMGWFRQAPGKEREFVAAISWRGSSMTSYADSVKGRFTISRDNARNTVYLQMNSLKPEDTAVYRCAAGHTYGATMTAPGSFDSWGQGTQVTVSS

SEQ ID NO: 28 - protein, 124 aa, Camelus sp.
QVQLQESGGGLVQAGGSLRLSCAASGRRTYPMGWFRQASGKERDFVAFISSSGRNTHYADSVKGRFTISRDNAKNAVYLQMNSLKPEDTAIYYCAVTLFHGSDYYTSASEYDYWGQGTQVAVSS

SEQ ID NO: 29 - protein, 125 aa, Camelus sp.
QVQLQESGGGLVQAGGSLRLSCAASGLYVSANDMGWFRQAPGKEREFVAAITWSGTDTTYADSVRGRFTISRDNARNTVYLQMNSLKPEDTGVYSCAAGHTYGAIMTAPGSFGSWGQGTQVTVSS

SEQ ID NO: 30 - protein, 123 aa, Camelus sp.
QVQLQESGGGLVQPGGSLRLSCAAKGSIFGINHMGWYRQGPGKQRELVAVTTSGGTINYANSVKGRFTISRDNAKNTVYLQMNSLKPEDTAVYYCNVKSHIPPNWGGPTPEYWGQGTQVTVSS

SEQ ID NO: 31 - protein, 127 aa, Camelus sp.
QVQLQESGGGLVQAGGSLRLSCAASGRTFDRYRLGWFRQVPGKEREFVAHISYNGANTYYADSVKGRFTVSRDNAKNTVYLQMNSLKPEDTAVYYCAATTSYLGGSYLYSPREYEYWGPGTQVTVSS

SEQ ID NO: 32 - protein, 125 aa, Camelus sp.
QVQLQESGGGLVQPGGSLRLSCAASGLWVSANDMGWFRQAPGKEREFVAAISWRGSSTSYADSVKGRFTISRDNARNTVRLQMNSLKPEDTAVYSCAAGHTHGATMTAPGNFDSWGQGTQVTVSS

SEQ ID NO: 33 - protein, 125 aa, Camelus sp.
QVQLQESGGGLVQAGGSLRLSCAASGLYVEANDMGWFRQAPGKEREFVAAIAWRGSATSYADSVKGRFTISRDNARNTVYLQMNSLKPEDTAVYSCASGHTHGATMTAPGSFGSWGQGTQVTVSS

SEQ ID NO: 34 - protein, 117 aa, Camelus sp.
QVQLQDSGGGLVQPGGSLRLSCVASGTIFSPDALGWYRQAPGKLREWVATINMGGDTNYVDSVKGRFTISRDNAKKTVYLQMDNLKPEDTAVYYCNLQSHYTSRIYWGQGTQVTVSS

SEQ ID NO: 35 - protein, 127 aa, Camelus sp.
QVQLQESGGGLVQAGGSLRLSCAASGRTFDRYRLGWFHQVPGKEREFVAHITYNGANTYYADSVKGRFTVSRDNAKNTVYLQMNSLKPEDTAVYYCAATTSYLGGSYLYSPREYEYWGPGTHVTVSS

SEQ ID NO: 36 - protein, 125 aa, Camelus sp.
QVQLQESGGGLVQAGGSLRLSCAASGLYVSANDMGWFRQAPGKEREFVATISWRGSSTSYVDSVKGRFTISRDNARNTVYLQMNSLKPEDTAVYSCAAGHSVGATMTAPGSFSSWGQGTQVTVSS

SEQ ID NO: 37 - protein, 117 aa, Camelus sp.
QVQLQDSGGGLVQPGGSLRLSCAASGTIFSPDVLGWYRQAPGKMREWVATIDGGGGTNYVDSVKGRFTISRDNAKKMLYLQMDNLKPEDTAVYYCNLQSHYTSRVYWGKGTQVTVSS

SEQ ID NO: 38 - protein, 125 aa, Camelus sp.
QVQLQESGGGLVQAGGSLRLSCAASGLWVSANDMGWFRQAPGKEREFVAAISWRGSSPSYLDSVKGRFTISRDNARNTVYLQMNSLKPEDTAVYSCAAGHTYGATMTAPGSFGSWGQGTQVTVSS

SEQ ID NO: 39 - protein, 125 aa, Camelus sp.
QVQLQESGGGLVQAGDSLRLSCAASGLYVSANDMGWFRQAPGKEREFVAAISWRGSSTSYADSVKGRFTISRDNARNTVYLQMNSLKPEDTAVYSCATGHTHGATMTAPGRFGSWGQGTQVTVSS

SEQ ID NO: 40 - protein, 125 aa, Camelus sp.
QVQLQESGGGLVQAGGSLRLSCAASGLYVSANDMGWFRQAPGKEREFVAAISWRGSSTSYGDSVKGRFTISRDNARNAVYLQMNSLKPEDTAVYSCAAGHTHGATMTAPGRFASWGQGTQVTVSS

SEQ ID NO: 41 - protein, 126 aa, Camelus sp.
QVQLQESGGALVQPGGSLRLSCAASGFTLDDYAIAWFRQAPGKEREGVSCTSSSDGYTYYVDSVKGRFTISRDHAKNMVYLQMNSLKAEDTAVYYCATEHAPAGYPCESVAEYDYWGQGTQVTVSS

SEQ ID NO: 42 - protein, 123 aa, Camelus sp.
QVQLQESGGGLVQPGGSLRLSCAASGSIFGINAMGWYRQAPGNQRELVAVITSGGTTTYANSVKGRFTISRDNAKNTVYLQMNSLKPEDTAVYYCNVKSHFPPNWGGPTPDYWGQGTQVTVSS

SEQ ID NO: 43 - protein, 112 aa, Camelus sp.
QVQLQESGGGLVQAGDSLRLSCAVSGRTFSRYAMGWFRQAPGKEREFVAGISWTGGNTYYADSVKGRFTISRDDAKKSLYLQMNSLKLEDTAVYYCNAYDGWGQGIQVTVSS

SEQ ID NO: 44 - protein, 126 aa, Camelus sp.
QVQLQESGGGLVQAGGSLRLSCAASGRTFSNYAMAWFRQAPGKEREFVAGITWTTSSTTYADSVKGRFTVSRDSAENTGYLQMNSLRPEDTALYYCATSRSTYSVNWYQYHDYNTWGQGTQVTVSS

SEQ ID NO: 45 - protein, 124 aa, Camelus sp.
QVQLQESGGGLVQTGGSLRLSCAASGRSFSSRAVGWFRQAPGKERDFVAAITWGGGGTHYADSVKGRFTISRDNARNTVYLQMNSLEPEDTAVYYCAACPIHYIGSDYSDYDFWGQGTQITVSS

SEQ ID NO: 46 - protein, 126 aa, Camelus sp.
QVQLQDSGGGLVQAGGSLRLSCAASGRTFSNYAMGWFRQAPGKEREFVAGITWTTGSTTYADSVKGRFTVSRDNAENTGYLQMNSLKPEDTAVYYCAASRATYSVNWYQYHDYNNWGQGTQVTVSS

SEQ ID NO: 47 - protein, 126 aa, Camelus sp.
QVQLQDSGGGLVQAGGSLRLSCAASGRTFSTYAMGWFRQAPGKEREFVAGITWTTGTTTYADSVKGRFTVSRDNAENTGYLQMNSLKPEDTAVYYCAASRATYSVNWYQYHDYNNWGQGTQVTVSS

SEQ ID NO: 48 - protein, 126 aa, Camelus sp.
QVQLQESGGGLVQAGGSLRLSCAASGRTFSSYAMGWFRQAPGKEREFVAGITWTTSSTTYADTVKGRFAVSRDTAENTGYLQMNSLKPEDTAVYHCAASRGTNSVNWYQYHDYYNWGQGTQVTVSS

SEQ ID NO: 49 - protein, 125 aa, Camelus sp.
QVQLQDSGGGLVQAGGSLRLSCAASGRTFSSEAMSWFRQAPGKEREIVGAISWSGGIYYADSVKGRFTISRDNAGNTVYLQMNSLKHTEDTAVYYCAAAGVSYYIGLRNGDYRVWGQGTQVTVSS

SEQ ID NO: 50 - protein, 126 aa, Camelus sp.
QVQLQESGGGLVQAGGSLRLSCAASGSIFSRYAIAWFRQAPGKEREFVAGITWNTGSTTYADSVKGRFTVSRDNAENTAYLQMNSLKPEDTAVYYCASSRAAYSVNWYQYHDYNTWGQGTQVTVSS

SEQ ID NO: 51 - protein, 126 aa, Camelus sp.
QVQLQESGGGLVQAGGSLRLSCAVSGRTFSNYAMGWFRQAPGKEREFVAGITWTTGSTTYADSVKGRFTVSRDNAENTGYLQMNSLKPEDTAVYYCAASRGTNSVNWYQYHDYNNWGQGTQVTVSS

SEQ ID NO: 52 - protein, 126 aa, Camelus sp.
QVQLQESGGGLVQAGGSLTLSCAASGRPFSSYAMGWFRQAPGKEREFVAGITWTTSSTTYADSVKGRFTVSRDIAKNTVYLQMNSLKPEDTAVYYCAASRATSSVNWYQYHDYNNWGQGTQVTVSS

SEQ ID NO: 53 - protein, 126 aa, Camelus sp.
QVQLQDSGGGLVQAGGSLRLSCAASGRTFSSYAMAWLRQTPGKEREFVAGITWATSSTTYTDSAKGRFTISRDSAQNTVYLQMNSLKSEDTAVYYCAASGPSNGIHWYRYSDYNNWGQGTQVTVSS

SEQ ID NO: 54 - protein, 126 aa, Camelus sp.
QVQLQDSGGGLVQAGGSLRLSCAASGRTFGSYAMGWFRQAPGKEREFVAGITWTTGSTTYADAVKGRFTVSRDNAENTAYLQMNFLRPEDTAVYYCAASRATYSVNWYQYHDYNNWGQGTQVTVSS

SEQ ID NO: 55 - protein, 126 aa, Camelus sp.
QVQLQESGGGLVQAGGSLRLSCAASGRTFSSYAMGWFRQAPGKEREFVAGITWTTGSTTYADSVKGRFTVSRDNAENTAYLQMNSLKPEDTAVYYCAASRAAYSVNWYQDHDYNNWGQGTQVTVSS

SEQ ID NO: 56 - protein, 126 aa, Camelus sp.
QVQLQESGGGLVQAGDSLNLSCAASGRTFSSYAVGWFRQAPGKEREFVAGITWATGSTTYADSVKGRFTVSRDNAKNTAYLHMNSLKPEDTAVYYCAASRAAYSVNWYQYHDYNTWGQGTQVTVSA

SEQ ID NO: 57 - protein, 126 aa, Camelus sp.
QVQLQESGGGLVQAGDSLRLSCAASGRTFSSYAMGWFRQAPGKEREFVAGITWTTGSTTYADSVKGRFTVSRDVVENTAYLQMNSLKPEDTALYYCATSRAAYSVNWYQYHDYNNWGQGTQVTVSS

SEQ ID NO: 58 - protein, 126 aa, Camelus sp.
QVQLQESGGGLVQAGGSLRLSCAASGRTFSSYAMGWFRQAPGKEREFVAGITWTTGSTTYADSVKGRFTVSRDNAENTGYLQMNSLKPEDTAVYNCAASRATMSVNWYQYHDYNNWGQGTQVTVSS

SEQ ID NO: 59 - protein, 126 aa, Camelus sp.
QVQLQESGGGLVQAGDSLRLSCAVSGRTFSNYAMGWFRQAPGKEREFVAGITWTTGSTTYADSVKGRFTVSRDNAENTGYLQMNSLKPEDTAVYYCAASRGTNSVNWYQYHDYNNWGQGTQVTVSS

SEQ ID NO: 60 - protein, 126 aa, Camelus sp.
QVQLQESGGGLVQAGGSLRLSCAASGRTFSSYAMAWFRQAPGKEREFVAGITWTTGSTTYADPVKGRYTVSRDNAENTGYLQMNSLKPEDSAVYYCTASRATNSVNWYQYHDYNNWGQGTQVTVSS

SEQ ID NO: 61 - protein, 126 aa, Camelus sp.
QVQLQESGGGLVQAGDSLRLSCAASGRSFSSYAMAWFRQAPGKEREFVAGITWTTSSTTYADSVKGRFTVSRDNARNTGYLRMNSLKPEDTAVYYCASSRGTNSVNWYQYHDYNNWGQGTQVTVSS

SEQ ID NO: 62 - protein, 124 aa, Camelus sp.
QVQLQDSGGGLVQTGGSLRLSCAASGRSFSSRAVGWFRQAPGKERDFVAAISWGGDGTHYADSVKGRFTISRDNARNTVYLQMNSLEPEDTAVYYCAAGTIYYIGSDYSDYNFWGQGTQVTVSS

SEQ ID NO: 63 - protein, 126 aa, Camelus sp.
QVQLQDSGGGLVQAGGSLRLSCAASGRTFSSYAMGWFRQAPGKEREFVAGITWTTGSTTYADSVKGRFTVYRDNAQNTVYLQMNRLKPEDTAVNYCAASRSTNSVNWYQYHDYNNWGQGNQVNVSS

SEQ ID NO: 64 - protein, 126 aa, Camelus sp.
QVQLQESGGGLVQAGGSLRLSCAASGRTFSSYAMAWFRQAPGKEREFVSGITWTTGSTTYADSVKGRFTVSRDNAENTAYLQMSSLKPEDTAVYYCAASRAANSVNWYQDHDYNVWGQGTQVTVSS

SEQ ID NO: 65 - protein, 126 aa, Camelus sp.
QVQLQESGGGLVQAGGSLRISCAASGRTFSNYAMGWFRQAPGKEREFVAGITWTTGSTTYADSVKGRFTVSRDNAENTAYLQMNSLKPEDTAVYYCAASRGTNSVNWYQYHDYNSWGQGTQVTVSS

SEQ ID NO: 66 - protein, 126 aa, Camelus sp.
QVQLQESGGGLVQAGASLSLSCEFSGRGFNNYAMGWFRQAPGKEREFVAGITWTTGSTTYADSVKGRFTVSRDNAENTMYLQMNSLEPEDTAVYYCAASRATDHVNWYQYRDYNNWGQGTQVTVSS

SEQ ID NO: 67 - protein, 123 aa, Camelus sp.
QVQLQDSGGGLVQTGGSLRLSCAASGRSFGSRAVGWFRQAPGKERDFVATISWGGGTHYADSVKGRFTISRDNARNTVYLEMNSLEPEDTAVYYCAAGPIHYISNDYSDYNFWGQGTQVTVSS

SEQ ID NO: 68 - protein, 131 aa, Camelus sp.
QVQLQESGGGLVQAGGSLRLSCTASGGAFDRYAMGWFRQAPGKEREFVAVISRGGLYTDYAESVKGRFTVYRDNAKNTVSLEMNSVKPDDTAIYSCAADLSAVGSRIEAGRARDSNEYDYWGQGTQVTVSS

SEQ ID NO: 69 - protein, 126 aa, Camelus sp.
QVQLQESGGGLVQAGGSLRLSCAASGRTFNRYAMGWFRQPPGKEREFVAGITWTTGSTTYADSVKARFTVSKDNAMNTGYLQMNSLKPEDTAVYYCAASRGINSVHWYEYQDYNNWGQGTQVTVSS

SEQ ID NO: 70 - protein, 123 aa, Camelus sp.
QVQLQDSGGGLVQAGESLRLSCKASGRTFSSYAMGWFRQAPGKERDFVAAISWSGTAYYADSVKGRFTISRDNAENMAYLQMNSLKPEDTAVYYCAAGGPYYIGTSYQEYDYWGQGTQITVSS

SEQ ID NO: 71 - protein, 123 aa, Camelus sp.
QVQLQESGGGLVQAGGSLRLSCAASGRTASSYAMGWFRQAPGKEREFVAAISWSHSTYYADSVKGRFTISRDNAENTVYLQMNSLKPEDTAVYSCAASGPYYIGTLSSEYDYWGQGTHVTVSS

SEQ ID NO: 72 - protein, 9 aa, Camelus sp.
FTFDDYAIG

SEQ ID NO: 73 - protein, 9 aa, Camelus sp.
RTFSSYAMG

SEQ ID NO: 74 - protein, 9 aa, Camelus sp.
RTFSSYAMG

SEQ ID NO: 75 - protein, 9 aa, Camelus sp.
RTFSTYAMG

SEQ ID NO: 76 - protein, 9 aa, Camelus sp.
RTFSSYAMG

SEQ ID NO: 77 - protein, 9 aa, Camelus sp.
RTVNSYAMA

SEQ ID NO: 78 - protein, 9 aa, Camelus sp.
RTFSTYAMG

SEQ ID NO: 79 - protein, 9 aa, Camelus sp.
RTFSSYAMG

SEQ ID NO: 80 - protein, 9 aa, Camelus sp.
RTFSSYAMG

SEQ ID NO: 81 - protein, 9 aa, Camelus sp.
RTFSTYAMG

SEQ ID NO: 82 - protein, 9 aa, Camelus sp.
LSLESTVVG

SEQ ID NO: 83 - protein, 9 aa, Camelus sp.
RTFSSYAMA

SEQ ID NO: 84 - protein, 9 aa, Camelus sp.
RTFSAYTVG

SEQ ID NO: 85 - protein, 9 aa, Camelus sp.
RTFSSYAMG

SEQ ID NO: 86 - protein, 9 aa, Camelus sp.
PTFSRYAMG

SEQ ID NO: 87 - protein, 9 aa, Camelus sp.
RTVSTSAMA

SEQ ID NO: 88 - protein, 9 aa, Camelus sp.
RTFSTYAMG

SEQ ID NO: 89 - protein, 9 aa, Camelus sp.
RTFSSYAMG

SEQ ID NO: 90 - protein, 12 aa, Camelus sp.
FTGYTFDDYAIG

SEQ ID NO: 91 - protein, 9 aa, Camelus sp.
RTFSTYAMG

SEQ ID NO: 92 - protein, 9 aa, Camelus sp.
RTYSSYAMG

SEQ ID NO: 93 - protein, 9 aa, Camelus sp.
FTFNDYAIG

SEQ ID NO: 94 - protein, 9 aa, Camelus sp.
LYVSANDMG

SEQ ID NO: 95 - protein, 7 aa, Camelus sp.
RRTYPMG

SEQ ID NO: 96 - protein, 9 aa, Camelus sp.
LYVSANDMG

SEQ ID NO: 97 - protein, 9 aa, Camelus sp.
SIFGINHMG

SEQ ID NO: 98 - protein, 9 aa, Camelus sp.
RTFDRYRLG

SEQ ID NO: 99 - protein, 9 aa, Camelus sp.
LWVSANDMG

SEQ ID NO: 100 - protein, 9 aa, Camelus sp.
LYVEANDMG

SEQ ID NO: 101 - protein, 9 aa, Camelus sp.
TIFSPDALG

SEQ ID NO: 102 - protein, 9 aa, Camelus sp.
RTFDRYRLG

SEQ ID NO: 103 - protein, 9 aa, Camelus sp.
LYVSANDMG

SEQ ID NO: 104 - protein, 9 aa, Camelus sp.
TIFSPDVLG

SEQ ID NO: 105 - protein, 9 aa, Camelus sp.
LWVSANDMG

SEQ ID NO: 106 - protein, 9 aa, Camelus sp.
LYVSANDMG

SEQ ID NO: 107 - protein, 9 aa, Camelus sp.
LYVSANDMG

SEQ ID NO: 108 - protein, 9 aa, Camelus sp.
FTLDDYAIA

SEQ ID NO: 109 - protein, 9 aa, Camelus sp.
SIFGINAMG

SEQ ID NO: 110 - protein, 9 aa, Camelus sp.
RTFSRYAMG

SEQ ID NO: 111 - protein, 9 aa, Camelus sp.
RTFSNYAMA

SEQ ID NO: 112 - protein, 9 aa, Camelus sp.
RSFSSRAVG

SEQ ID NO: 113 - protein, 9 aa, Camelus sp.
RTFSNYAMG

SEQ ID NO: 114 - protein, 9 aa, Camelus sp.
RTFSTYAMG

SEQ ID NO: 115 - protein, 9 aa, Camelus sp.
RTFSSYAMG

SEQ ID NO: 116 - protein, 9 aa, Camelus sp.
RTFSSEAMS

SEQ ID NO: 117 - protein, 9 aa, Camelus sp.
SIFSRYAIA

SEQ ID NO: 118 - protein, 9 aa, Camelus sp.
RTFSNYAMG

SEQ ID NO: 119 - protein, 9 aa, Camelus sp.
RPFSSYAMG

SEQ ID NO: 120 - protein, 9 aa, Camelus sp.
RTFSSYAMA

SEQ ID NO: 121 - protein, 9 aa, Camelus sp.
RTFGSYAMG

SEQ ID NO: 122 - protein, 9 aa, Camelus sp.
RTFSSYAMG

SEQ ID NO: 123 - protein, 9 aa, Camelus sp.
RTFSSYAVG

SEQ ID NO: 124 - protein, 9 aa, Camelus sp.
RTFSSYAMG

SEQ ID NO: 125 - protein, 9 aa, Camelus sp.
RTFSSYAMG

SEQ ID NO: 126 - protein, 9 aa, Camelus sp.
RTFSNYAMG

SEQ ID NO: 127 - protein, 9 aa, Camelus sp.
RTFSSYAMA

SEQ ID NO: 128 - protein, 9 aa, Camelus sp.
RSFSSYAMA

SEQ ID NO: 129 - protein, 9 aa, Camelus sp.
RSFSSRAVG

SEQ ID NO: 130 - protein, 9 aa, Camelus sp.
RTFSSYAMG

SEQ ID NO: 131 - protein, 9 aa, Camelus sp.
RTFSSYAMA

SEQ ID NO: 132 - protein, 9 aa, Camelus sp.
RTFSNYAMG

SEQ ID NO: 133 - protein, 9 aa, Camelus sp.
RGFNNYAMG

SEQ ID NO: 134 - protein, 9 aa, Camelus sp.
RSFGSRAVG

SEQ ID NO: 135 - protein, 9 aa, Camelus sp.
GAFDRYAMG

SEQ ID NO: 136 - protein, 9 aa, Camelus sp.
RTFNRYAMG

SEQ ID NO: 137 - protein, 9 aa, Camelus sp.
RTFSSYAMG

SEQ ID NO: 138 - protein, 9 aa, Camelus sp.
RTASSYAMG

SEQ ID NO: 139 - protein, 16 aa, Camelus sp.
CLKSSDGRIYYADSVK

SEQ ID NO: 140 - protein, 16 aa, Camelus sp.
GISGSGGRTYYEDSVK

SEQ ID NO: 141 - protein, 16 aa, Camelus sp.
AISWSGDSTNYADSVK

SEQ ID NO: 142 - protein, 16 aa, Camelus sp.
GISGSGGRTYYEDSVK

SEQ ID NO: 143 - protein, 16 aa, Camelus sp.
AISFSGDSTAYADSVK

SEQ ID NO: 144 - protein, 16 aa, Camelus sp.
ATTWRGTSTNYADPVK

SEQ ID NO: 145 - protein, 16 aa, Camelus sp.
GISGSGGRTYYEDSVK

SEQ ID NO: 146 - protein, 16 aa, Camelus sp.
TINWSGGTTYYADSVK

SEQ ID NO: 147 - protein, 16 aa, Camelus sp.
AISFSGDSTDYADSVK

SEQ ID NO: 148 - protein, 16 aa, Camelus sp.
GISGSGGRTYYEDSVK

SEQ ID NO: 149 - protein, 16 aa, Camelus sp.
CISSGDTNTLYVDSVK

SEQ ID NO: 150 - protein, 16 aa, Camelus sp.
TINWSGSSTYYVDSVK

SEQ ID NO: 151 - protein, 16 aa, Camelus sp.
TVNWSGGSSYYADSVK

SEQ ID NO: 152 - protein, 16 aa, Camelus sp.
GISGSGGRTYYEDSVK

SEQ ID NO: 153 - protein, 16 aa, Camelus sp.
AISWSGDRTDYAESVK

SEQ ID NO: 154 - protein, 16 aa, Camelus sp.
SISWTGDSTNYADSVR

SEQ ID NO: 155 - protein, 16 aa, Camelus sp.
GISGSGGRTYYEDSVK

SEQ ID NO: 156 - protein, 16 aa, Camelus sp.
GISGSGGRTYYEDSVK

SEQ ID NO: 157 - protein, 16 aa, Camelus sp.
CISSSDGTTYYADSVK

SEQ ID NO: 158 - protein, 16 aa, Camelus sp.
GISGSGGRTYYEDSVK

SEQ ID NO: 159 - protein, 16 aa, Camelus sp.
AINWSGGTTYYTDSVK

SEQ ID NO: 160 - protein, 16 aa, Camelus sp.
WISSSDGSTKYADSVK

SEQ ID NO: 161 - protein, 17 aa, Camelus sp.
AISWRGSSMTSYADSVK

SEQ ID NO: 162 - protein, 16 aa, Camelus sp.
FISSSGRNTHYADSVK

SEQ ID NO: 163 - protein, 16 aa, Camelus sp.
AITWSGTDTTYADSVR

SEQ ID NO: 164 - protein, 15 aa, Camelus sp.
VTTSGGTINYANSVK

SEQ ID NO: 165 - protein, 16 aa, Camelus sp.
HISYNGANTYYADSVK

SEQ ID NO: 166 - protein, 16 aa, Camelus sp.
AISWRGSSTSYADSVK

SEQ ID NO: 167 - protein, 16 aa, Camelus sp.
AIAWRGSATSYADSVK

SEQ ID NO: 168 - protein, 15 aa, Camelus sp.
TINMGGDTNYVDSVK

SEQ ID NO: 169 - protein, 16 aa, Camelus sp.
HITYNGANTYYADSVK

SEQ ID NO: 170 - protein, 16 aa, Camelus sp.
TISWRGSSTSYVDSVK

SEQ ID NO: 171 - protein, 15 aa, Camelus sp.
TIDGGGGTNYVDSVK

SEQ ID NO: 172 - protein, 16 aa, Camelus sp.
AISWRGSSPSYLDSVK

SEQ ID NO: 173 - protein, 16 aa, Camelus sp.
AISWRGSSTSYADSVK

SEQ ID NO: 174 - protein, 16 aa, Camelus sp.
AISWRGSSTSYGDSVK

SEQ ID NO: 175 - protein, 16 aa, Camelus sp.
CTSSSDGYTYYVDSVK

SEQ ID NO: 176 - protein, 15 aa, Camelus sp.
VITSGGTTTYANSVK

SEQ ID NO: 177 - protein, 16 aa, Camelus sp.
GISWTGGNTYYADSVK

SEQ ID NO: 178 - protein, 16 aa, Camelus sp.
GITWTTSSTTYADSVK

SEQ ID NO: 179 - protein, 16 aa, Camelus sp.
AITWGGGGTHYADSVK

SEQ ID NO: 180 - protein, 16 aa, Camelus sp.
GITWTTGSTTYADSVK

SEQ ID NO: 181 - protein, 16 aa, Camelus sp.
GITWTTGTTTYADSVK

SEQ ID NO: 182 - protein, 16 aa, Camelus sp.
GITWTTSSTTYADTVK

SEQ ID NO: 183 - protein, 15 aa, Camelus sp.
AISWSGGIYYADSVK

SEQ ID NO: 184 - protein, 16 aa, Camelus sp.
GITWNTGSTTYADSVK

SEQ ID NO: 185 - protein, 16 aa, Camelus sp.
GITWTTGSTTYADSVK

SEQ ID NO: 186 - protein, 16 aa, Camelus sp.
GITWTTSSTTYADSVK

SEQ ID NO: 187 - protein, 16 aa, Camelus sp.
GITWATSSTTYTDSAK

SEQ ID NO: 188 - protein, 16 aa, Camelus sp.
GITWTTGSTTYADAVK

SEQ ID NO: 189 - protein, 16 aa, Camelus sp.
GITWTTGSTTYADSVK

SEQ ID NO: 190 - protein, 16 aa, Camelus sp.
GITWATGSTTYADSVK

SEQ ID NO: 191 - protein, 16 aa, Camelus sp.
GITWTTGSTTYADSVK

SEQ ID NO: 192 - protein, 16 aa, Camelus sp.
GITWTTGSTTYADSVK

SEQ ID NO: 193 - protein, 16 aa, Camelus sp.
GITWTTGSTTYADSVK

SEQ ID NO: 194 - protein, 16 aa, Camelus sp.
GITWTTGSTTYADPVK

SEQ ID NO: 195 - protein, 16 aa, Camelus sp.
GITWTTSSTTYADSVK

SEQ ID NO: 196 - protein, 16 aa, Camelus sp.
AISWGGDGTHYADSVK

SEQ ID NO: 197 - protein, 16 aa, Camelus sp.
GITWTTGSTTYADSVK

SEQ ID NO: 198 - protein, 16 aa, Camelus sp.
GITWTTGSTTYADSVK

SEQ ID NO: 199 - protein, 16 aa, Camelus sp.
GITWTTGSTTYADSVK

SEQ ID NO: 200 - protein, 16 aa, Camelus sp.
GITWTTGSTTYADSVK

SEQ ID NO: 201 - protein, 15 aa, Camelus sp.
TISWGGGTHYADSVK

SEQ ID NO: 202 - protein, 16 aa, Camelus sp.
VISRGGLYTDYAESVK

SEQ ID NO: 203 - protein, 16 aa, Camelus sp.
GITWTTGSTTYADSVK

SEQ ID NO: 204 - protein, 15 aa, Camelus sp.
AISWSGTAYYADSVK

SEQ ID NO: 205 - protein, 15 aa, Camelus sp.
AISWSHSTYYADSVK

SEQ ID NO: 206 - protein, 17 aa, Camelus sp.
ERKFYGLGYCSEYGMDY

SEQ ID NO: 207 - protein, 18 aa, Camelus sp.
NLRGYNSGTYYRASDFDS

SEQ ID NO: 208 - protein, 17 aa, Camelus sp.
DSHYGYSEYIRGLGMDY

SEQ ID NO: 209 - protein, 18 aa, Camelus sp.
KPRGYNSGTYYRASDFDS

SEQ ID NO: 210 - protein, 17 aa, Camelus sp.
DSHYGYSEYIRGLGMDY

SEQ ID NO: 211 - protein, 15 aa, Camelus sp.
NTYYRGGGYLTEYNL

SEQ ID NO: 212 - protein, 18 aa, Camelus sp.
KPRGYNSVTYYRASDFDS

SEQ ID NO: 213 - protein, 17 aa, Camelus sp.
VRYGGGGFFTDSKSFDY

SEQ ID NO: 214 - protein, 17 aa, Camelus sp.
DSHYGYSEYIRGLGMDY

SEQ ID NO: 215 - protein, 18 aa, Camelus sp.
KPRGYNSGTYYRASDFDS

SEQ ID NO: 216 - protein, 15 aa, Camelus sp.
GVDSGYGCPAYEYDS

SEQ ID NO: 217 - protein, 17 aa, Camelus sp.
SHYGGSGYLLEQRSYAY

SEQ ID NO: 218 - protein, 17 aa, Camelus sp.
TEYGGSGYLLGGLDYHY

SEQ ID NO: 219 - protein, 18 aa, Camelus sp.
NTRGYNSGTYYRASDFDS

SEQ ID NO: 220 - protein, 17 aa, Camelus sp.
DSHYGYSEYIRGLGMDY

SEQ ID NO: 221 - protein, 17 aa, Camelus sp.
GSGIYAYLYTDHRAYEY

SEQ ID NO: 222 - protein, 18 aa, Camelus sp.
KPRGYNSGTYYRASDFDF

SEQ ID NO: 223 - protein, 18 aa, Camelus sp.
NTRGYNSGSYYRASDFDS

SEQ ID NO: 224 - protein, 17 aa, Camelus sp.
DLFTSYCSDPMHPRYDY

SEQ ID NO: 225 - protein, 19 aa, Camelus sp.
ANPRGYNSGTYYRASDFDS

SEQ ID NO: 226 - protein, 17 aa, Camelus sp.
TYYGGGGFFTESHQYDY

SEQ ID NO: 227 - protein, 18 aa, Camelus sp.
AQLFAGSYYYTAPEWYDY

SEQ ID NO: 228 - protein, 16 aa, Camelus sp.
GHTYGATMTAPGSFDS

SEQ ID NO: 229 - protein, 17 aa, Camelus sp.
TLFHGSDYYTSASEYDY

SEQ ID NO: 230 - protein, 16 aa, Camelus sp.
GHTYGAIMTAPGSFGS

SEQ ID NO: 231 - protein, 15 aa, Camelus sp.
KSHIPPNWGGPTPEY

SEQ ID NO: 232 - protein, 18 aa, Camelus sp.
TTSYLGGSYLYSPREYEY

SEQ ID NO: 233 - protein, 16 aa, Camelus sp.
GHTHGATMTAPGNFDS

SEQ ID NO: 234 - protein, 16 aa, Camelus sp.
GHTHGATMTAPGSFGS

SEQ ID NO: 235 - protein, 9 aa, Camelus sp.
QSHYTSRIY

SEQ ID NO: 236 - protein, 18 aa, Camelus sp.
TTSYLGGSYLYSPREYEY

SEQ ID NO: 237 - protein, 16 aa, Camelus sp.
GHSVGATMTAPGSFSS

SEQ ID NO: 238 - protein, 9 aa, Camelus sp.
QSHYTSRVY

SEQ ID NO: 239 - protein, 16 aa, Camelus sp.
GHTYGATMTAPGSFGS

SEQ ID NO: 240 - protein, 16 aa, Camelus sp.
GHTHGATMTAPGRFGS

SEQ ID NO: 241 - protein, 16 aa, Camelus sp.
GHTHGATMTAPGRFAS

SEQ ID NO: 242 - protein, 17 aa, Camelus sp.
EHAPAGYPCESVAEYDY

SEQ ID NO: 243 - protein, 15 aa, Camelus sp.
KSHFPPNWGGPTPDY

SEQ ID NO: 244 - protein, 3 aa, Camelus sp.
YDG

SEQ ID NO: 245 - protein, 17 aa, Camelus sp.
SRSTYSVNWYQYHDYNT

SEQ ID NO: 246 - protein, 15 aa, Camelus sp.
CPIHYIGSDYSDYDF

SEQ ID NO: 247 - protein, 15 aa, Camelus sp.
ATYSVNWYQYHDYNN

SEQ ID NO: 248 - protein, 17 aa, Camelus sp.
SRATYSVNWYQYHDYNN

SEQ ID NO: 249 - protein, 17 aa, Camelus sp.
SRGTNSVNWYQYHDYYN

SEQ ID NO: 250 - protein, 16 aa, Camelus sp.
AGVSYYIGLRNGDYRV

SEQ ID NO: 251 - protein, 17 aa, Camelus sp.
SRAAYSVNWYQYHDYNT

SEQ ID NO: 252 - protein, 17 aa, Camelus sp.
SRGTNSVNWYQYHDYNN

SEQ ID NO: 253 - protein, 17 aa, Camelus sp.
SRATSSVNWYQYHDYNN

SEQ ID NO: 254 - protein, 17 aa, Camelus sp.
SGPSNGIHWYRYSDYNN

SEQ ID NO: 255 - protein, 17 aa, Camelus sp.
SRATYSVNWYQYHDYNN

SEQ ID NO: 256 - protein, 17 aa, Camelus sp.
SRAAYSVNWYQDHDYNN

SEQ ID NO: 257 - protein, 17 aa, Camelus sp.
SRAAYSVNWYQYHDYNT

SEQ ID NO: 258 - protein, 17 aa, Camelus sp.
SRAAYSVNWYQYHDYNN

SEQ ID NO: 259 - protein, 17 aa, Camelus sp.
SRATMSVNWYQYHDYNN

SEQ ID NO: 260 - protein, 17 aa, Camelus sp.
SRGTNSVNWYQYHDYNN

SEQ ID NO: 261 - protein, 17 aa, Camelus sp.
SRATNSVNWYQYHDYNN

SEQ ID NO: 262 - protein, 17 aa, Camelus sp.
SRGTNSVNWYQYHDYNN

SEQ ID NO: 263 - protein, 15 aa, Camelus sp.
GTIYYIGSDYSDYNF

SEQ ID NO: 264 - protein, 17 aa, Camelus sp.
SRSTNSVNWYQYHDYNN

SEQ ID NO: 265 - protein, 17 aa, Camelus sp.
SRAANSVNWYQDHDYNV

SEQ ID NO: 266 - protein, 17 aa, Camelus sp.
SRGTNSVNWYQYHDYNS

SEQ ID NO: 267 - protein, 17 aa, Camelus sp.
SRATDHVNWYQYRDYNN

SEQ ID NO: 268 - protein, 15 aa, Camelus sp.
GPIHYISNDYSDYNF

SEQ ID NO: 269 - protein, 22 aa, Camelus sp.
DLSAVGSRIEAGRARDSNEYDY

SEQ ID NO: 270 - protein, 17 aa, Camelus sp.
SRGINSVHWYEYQDYNN

SEQ ID NO: 271 - protein, 15 aa, Camelus sp.
GGPYYIGTSYQEYDY

SEQ ID NO: 272 - protein, 15 aa, Camelus sp.
SGPYYIGTLSSEYDY

(19) United States
(12) Patent Application Publication
(10) Pub. No.: US 2011/0166076 A1
(43) Pub. Date: Jul. 7, 2011

(54) Antigen-binding proteins that inhibit superantigens for the treatment of skin diseases

(75) Inventors: Hendrik Adams, Vleuten (NL); Bram Theodorus Hendrikus Maassen, Nijmegen (NL)

(73) Assignee: BAC IP B.V., Naarden (NL)

(21) Appl. No.: 13/063,363

(22) PCT Filed: Sep. 10, 2009
(86) PCT No.: PCT/NL2009/050545

§ 371 Date: Mar. 10, 2011

Related U.S. Application Data
(60) Provisional application No. 61/095,770, filed on Sep. 10, 2008.

(30) Foreign Application Priority Data
Sep. 10, 2008 (EP) 08164044.3

(51) Int. Cl. A61K 38/00; C07K 14/00; A61P 17/00; C07K 16/00

(52) U.S. Cl. 514/18.6; 530/350; 530/387.1

(57) ABSTRACT

The present invention relates to superantigen-specificantigen-binding proteins comprising an immunoglobulin-derived variable domain that comprises a complete antigen binding site for an epitope on the superantigen in a single polypeptide chain. The antigen-binding proteins of the invention may be used in the treatment skin diseases. The antigen-binding proteins of the invention may be used in compositions for topical administration.

### FIELD OF THE INVENTION

The present invention relates to the fields of medicin, immunology and molecular biology. The invention provides compositions for use in the treatment of conditions that are related to the exposure to superantigens produced during infection with bacterial pathogens such as Staphylococcus aureus and Streptococcus pyogenes, for example skin disorders.

### BACKGROUND OF THE INVENTION

Bacterial and viral superantigens (SAg), such as bacterial SAgs produced by Staphylococcus aureus and Streptococcus pyogenes, are potent T-cell stimulatory protein molecules and can cause massive overstimulation of the host immune system through cytokine release, T cell proliferation and T cell anergy and apoptosis. SAgs are produced intracellularly and are released upon infection as extracellular mature toxins, where they are capable of activating up to 20% of the T cells of the body.

The superantigenic activity has been attributed to the ability to bind simultaneously to Major Histocompatibility Complex Class II (MHC class II) receptors and the Vβ regions of T cell receptors. The formation of such a trimolecular complex results in the activation of a large population of T cells, ultimately releasing a massive amount of inflammatory cytokines. With no adequate treatment, this overstimulation of the immune system can eventually lead to a systemic response known as Toxic Shock Syndrome (TSS), and this condition is regarded to be among the most life-threatening syndromes affecting humans. Examples of other illnesses or conditions that are caused or aggravated by bacterial superantigens are sepsis, food poisoning (e.g. Staphylococcal food poisoning) and atopic dermatitis (AD).

Present therapy is primarily supportive, with administration of fluids, plasmapheresis using various adsorption devices, antibiotics, immunosuppression to prevent T cell activation and release of cytokines, vasopressor agents, corticosteroids, synthetic antibodies and peptides to mimic SAg binding regions on MHC Class II and/or on the T cell receptors, thereby blocking interaction and preventing T cell activation. Although these therapies can result in beneficial effect, none of them is actually capable of curing the disease. In plasmapheresis toxic components are removed together with the contaminated plasma, which has been shown to result in beneficial effects in severe cases of sepsis (Scharfman et al., 1979 N Engl J Med. 300(22):1277-8; Drenger et al., 1985, Lancet. 2(8461):943). However, in plasmapheresis also beneficial components are removed.

More recently various adsorption devices have been developed that were shown to bind to cytokines, enterotoxins and TSST-1, which is the causative enterotoxin for TSS. Animal studies showed that use of adsorption devices had inhibitory effects on inflammatory cytokine production and reduced mortality (Miwa et al., 2003 Int J Infect Dis. 7(1):21-6; Taniguchi et al., 2006, Crit Care Med. 34(3):800-6; Fenwick et al., 2003, Crit Care Med. 31(1):171-8; Miwa et al., 2006 Blood Purif. 24(3):319-26). These adsorption devices are, however, based on polystyrene and are therefore aspecific in the sense that they bind a poorly defined variety of small proteins, which include SAgs but which may also include beneficial molecules such as cytokines.

Goldman et al. (2006, Anal. Biochem. 78: 8245-8255) disclose single domain antibodies against various viral and bacterial toxins, including Staphylococcal enterotoxin B. These single domain antibodies are obtained from a semi-synthetic Llama library for use in immunoassay formats such as sandwich assays. However, the single domain antibodies obtained from semi-synthetic library show reduced overall affinities for the toxins, which render them unsuitable for use in therapeutic applications.

Currently, severe AD is generally treated with antimicrobial medications along with topical administration of corticosteroids. Examples of antimicrobial medications that are used in the treatment of AD include erythromycin, azithromycin, clarithromycin, cephalosporin cefuroxime axetil, cephalexin, trimethoprim-sulfamethoxazole (co-trimoxazole), clyndamycin and penicillinase-resistant penicillins such as dicloxacillin, oxacillin, and cloxacillin. The increasing prevalence of MRSA in the community has made treatment options more difficult for some patients. In these patients, clyndamycin, fusidic acid or combination therapy with trimethoprim-sulfamethoxazole and intranasal mupirocin can be effective in eliminating MRSA infection, at least until MRSA also becomes resistant to these therapies.

It has been found that some patients are not responding satisfactorily to corticosteroid treatment. A potential reason for this observation may be that SAgs have been shown to induce corticosteroid resistance and would thereby increase the severity of AD (Hauk et al. [2000] J Allergy Clin Immunol 105:782-787). The deleterious effect of SAgs on atopic skin begins with S. aureus colonization and infection. Reducing the avidity of atopic skin for S. aureus colonization begins with averting the SAg driven allergic skin inflammation. Once S. aureus attaches and secretes its toxins, a powerful immune response ensues, requiring a different approach to therapy.

In light of the above, there is still a need for adsorption systems with a higher specificity for the superantigen target molecules. More generally it is an object of the present invention to provide for novel therapeutic strategies, compositions and medicaments in the treatment and prevention of illnesses or conditions that are caused by SAgs. In addition, the emergence of multi-drug resistant bacteria and the lack of response to treatment with corticosteroids alone drive the exploration of new options for therapy for skin disorders, for example for AD or related skin diseases.

### DESCRIPTION OF THE INVENTION

In a first aspect, the present invention relates to antigen-binding proteins that specifically bind to a superantigen. We will refer to such antigen-binding proteins as “superantigen-binding proteins” or “SAg-binding proteins”.

A “superantigen” or “SAg” is herein understood to be a substance, normally a protein of microbial origin, that binds to major histocompatibility complex (MHC) Class II molecules and stimulates T-cell, via interaction with the Vβ domain of the T-cell receptor (TCR). SAgs have the particular characteristic of being able to interact with a large proportion of the T-cell repertoire, i.e. all the members of a given Vβ subset or “family”, or even with more than one Vβ subset, rather than with single, molecular clones from distinct Vβ families as is the case with a conventional (MHC-restricted) antigen. A superantigen has a mitogenic effect that is MHC Class II dependent but MHC-unrestricted. SAgs require cells that express MHC Class II for stimulation of T-cells to occur. A SAg is herein thus also understood to mean a substance that has SAg activity. “Superantigen activity” or “SAg activity” signifies a capacity to stimulate T-cells in an MHC-Class II-dependent but MHC-unrestricted manner. In the context of the invention, SAg activity can be detected directly by measuring specific expansion of activated T-cells bearing a particular Vβ-chain, or indirectly in a functional assay by measuring IL-2 release by activated T-cells. A suitable assay for determining the activity of SAg's is e.g. a proliferation assay as described by Visvanathan et al. (2001, Infect Immun. 69(2):875-84). Briefly, in this assay human peripheral blood mononuclear cells (PBMCs) are place in 96-well titer plates and stimulated with various doses of SAg toxin or with a combination of the fixed dose of SAg toxin and different doses of antibody. The cells are incubated for 6 days, and proliferation of the cells is measured via tritiated thymidine incorporation.

In a preferred embodiment, the SAg-binding proteins of the invention are antibodies, more preferably such antibodies or fragments thereof are derived from antibodies naturally devoid of light chains. Antibodies naturally devoid of light chains may be obtained e.g. by immunisation of camelids (e.g. llama's) or sharks (see further below). These antibodies comprise heavy chains only and are devoid of light chains. The advantage of use of such single domain heavy chain antibodies is that they are exceptionally stable even at higher temperatures, small and are easily produced in microbial host organisms such as Saccharomyces cerevisiae. Thus, a SAg-binding protein of the invention preferably comprises an immunoglobulin-derived variable domain that comprises a complete antigen-binding site for an epitope on a target molecule in a single polypeptide chain. Such SAg-binding proteins specifically include but are not limited to:

1) antibodies obtainable from camelids and sharks that consist of only heavy chains and that are naturally devoid of light chains;

2) variable domains of the antibodies defined in 1), usually referred to as VHH domains;

3) engineered forms of the antibodies defined in 1) or domains in 2) such as e.g. “camelidised” antibodies in which frame work sequences of a camelid (or shark) VHH domain are grafted with CDRs obtained from other sources;

4) engineered forms of immunoglobuline-like variable domains in which frame works sequences from a variety of immunoglobuline-like molecules are combined with CDRs specific for a given target molecule as e.g. described in WO 04/108749.

In a preferred SAg-binding protein of the invention, the single polypeptide chain of the variable domain that comprises the full antigen-binding capacity preferably has an amino acid sequence and structure that can be considered to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. These framework regions and complementary determining regions are preferably operably linked in the order FR1-CDR1-FR2-CDR2-FR3-CDR3-FR4 (from amino terminus to carboxy terminus).

The total number of amino acid residues in the variable domain with full antigen-binding capacity can be in the region of 110-135, and preferably is in the region of 115-129. However, a variable domain with full antigen-binding capacity in accordance with the invention is not particularly limited as to its length and/or size, as the domain meets the further functional requirements outlined herein and/or is suitable for the purposes described herein. The amino acid residues of a variable domain with full antigen-binding capacity are numbered according to the general numbering for VH domains given by Kabat et al. (“Sequence of proteins of immunological interest”, US Public Health Services, NIH Bethesda, MD., Publication No. 91), as applied to VHH domains from Camelids by Riechmann and Muyldermans (1999, J. Immunol. Methods 231 (1-2): 25-38, see for example FIG. 2 of said reference) and by Harmsen et al. (2000, Molecular Immunology 37: 579-590, see for example FIG. 1 of said reference).

According to this numbering, in a variable domain with full antigen-binding capacity: FR1 comprises the amino acid residues at positions 1-26; CDR1 comprises the amino acid residues at positions 27-35; FR2 comprises the amino acids at positions 36-49; CDR2 comprises the amino acid residues at positions 50-64; FR3 comprises the amino acid residues at positions 65-94; CDR3 comprises the amino acid residues at positions 95-102; and, finally, FR4 comprises the amino acid residues at positions 103-113.

In this respect, it should be noted that—as is well known in the art for VH domains and for VHH domains—the total number of amino acid residues in each of the CDR's may vary and may not correspond to the total number of amino acid residues indicated by the Kabat numbering. However, based on the conserved amino acids of the frame work region a skilled person will be able to align the respective frame work and complementarity determining regions in accordance with the Kabat definitions for those variable domains with full antigen-binding capacity that have a length other than 113 amino acids. Examples thereof are given in the sequence listings SEQ ID NO's: 5 -71. Alternative methods for numbering the amino acid residues of VH domains, which methods can also be applied in an analogous manner to VHH domains from Camelids and to variable domains with full antigen-binding capacity, are the method described by Chothia et al. (Nature 342, 877-883 (1989)), the so-called “AbM definition” and the so-called “contact definition”, or the IMGT numbering system (Lefranc et al., 1999, Nucl. Acids Res. 27: 209-212).

In a preferred SAg-binding protein of the invention, the frame work amino acid sequence of a variable domain with full antigen-binding capacity preferably has at least 50, 55, 60, 65, 70, 75, 80, 85, 90, 95 or 100% amino acid identity with the frame work amino acid sequence of any one of SEQ ID No's: 5-71.

More preferably, the amino acid residues that are present at each position (according to the Kabat numbering) of the FR1, FR2, FR3 and FR4 of the single polypeptide chain of the variable domain that comprises the full antigen-binding capacity preferably are as indicated in Tables 1 to 4 for FR1, FR2, FR3 and FR4. Thereby preferably the frame work amino acid residues of a variable domain with full antigen-binding capacity are chosen from the non-limiting residues in Tables 1 to 4 that can be present at each position (according to the Kabat numbering) of the FR1, FR2, FR3 and FR4 of naturally occurring Camelid VHH domains (data was taken from patent WO 2006/040153 PCT/EP2005/011018). More preferably, however, the frame work amino acid residues of a variable domain with full antigen-binding capacity are chosen from the amino acid residues in Tables 1 to 4 that are present at each position (according to the Kabat numbering) of the FR1, FR2, FR3 and FR4 of the amino acid sequences of any one of SEQ ID No's: 1-4 of antigen-binding proteins that specifically bind a SAg. For each position, the amino acid residue that most frequently occurs at each position is indicated in bold in Tables 1 to 4.

Thus, in a preferred embodiment of the invention, on the basis of the amino acid residues present on the positions described in Tables 1 to 4, the amino acid sequence of a variable domain comprising the full antigen-binding capacity in a SAg-binding protein of the invention can have the structure:

FR1-CDR1-FR2-CDR2-FR3-CDR3-FR4

in which FR1 has an amino acid sequence chosen from the group consisting of:

| | | |
|---|---|---|
| a) | [1] QVQLQESGGGLVQAGGSLRLSCAASG [26]; | (SEQ ID: 1) |

b) an amino acid sequence that has at least 50, 55, 60, 65, 70, 75, 80, 85, 90, 95 or 100% sequence identity with the sequence in a); and/or,
c) the amino acid sequence of a) that has one or more amino acid substitutions as defined in Table 1;
   in which FR2 is chosen from the group consisting of the amino acid sequence:

| | | |
|---|---|---|
| d) | [36] WFRQAPGKEREFVA [49]; | (SEQ ID: 2) |

e) an amino acid sequence that has at least 50, 55, 60, 65, 70, 75, 80, 85, 90, 95 or 100% sequence identity with the sequence in d); and/or
f) the amino acid sequence of d) that has one or more amino acid substitutions as defined in Table 2;
   in which FR3 is chosen from the group consisting of the amino acid sequence:

(SEQ ID: 3)

| | |
|---|---|
| g) | [65] GRFTISRDNAKNTVYLQMNSLKPEDTAVYYCAA [94]; |

h) an amino acid sequence that has at least 50, 55, 60, 65, 70, 75, 80, 85, 90, 95 or 100% sequence identity with the sequence in g); and/or,
i) the amino acid sequence of g) that has one or more amino acid substitutions as defined in Table 3; and,
   in which FR4 is chosen from the group consisting of the amino acid sequence:

| | | |
|---|---|---|
| j) | [103] WGQGTQVTVSS [113]; | (SEQ ID: 4) |

k) an amino acid sequence that has at least 50, 55, 60, 65, 70, 75, 80, 85, 90, 95 or 100% sequence identity with the sequence in j); and/or,
l) the amino acid sequence of j) that has one or more amino acid substitutions as defined in Table 4.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by various methods, known to those skilled in the art. In a preferred embodiment, sequence identity is determined by comparing the whole length of the sequences as identified herein.

Preferred methods to determine identity are designed to give the largest match between the sequences tested. Methods to determine identity and similarity are codified in publicly available computer programs. Preferred computer program methods to determine identity and similarity between two sequences include e.g. the BestFit, BLASTP, BLASTN, and FASTA (Altschul, S. F. et al., J. Mol. Biol. 215:403-410 (1990), publicly available from NCBI and other sources (BLAST Manual, Altschul, S., et al., NCBI NLM NIH Bethesda, Md. 20894). A most preferred algorithm used is EMBOSS (http://www.ebi.ac.uk/emboss/align). Preferred parameters for amino acid sequences comparison using EMBOSS are gap open 10.0, gap extend 0.5, Blosum 62 matrix. Preferred parameters for nucleic acid sequences comparison using EMBOSS are gap open 10.0, gap extend 0.5, DNA full matrix (DNA identity matrix).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

The SAg-binding protein of the invention is a component that specifically binds to the target molecule with a desired binding affinity (as herein defined). The SAg-binding protein of the invention preferably is a mono-specific antigen-binding protein. A composition comprising a mono-specific antigen-binding protein, such as the immunoadsorbant materials of the present invention, is understood to mean a composition having a homogeneous population of the SAg-binding protein. It follows that the mono-specific SAg-binding protein is specific for a single epitope or ligand. It is however expressly included in the invention that the immunoadsorbant material may comprise more than one type of mono-specific SAg-binding protein, each consisting of a homogeneous population. Usually, however, in the context of the present invention, an immunoadsorbant material will not comprise more than 4, 6, 8, 10 or 20 different mono-specific SAg-binding proteins. The SAg-binding protein will usually be an antibody or fragment thereof, in which case the mono-specific SAg-binding protein will thus be a monoclonal antibody or a fragment thereof, which may be obtained from a cloned cell-line (e.g. hybridoma) or expressed from a cloned coding sequence. The term mono-specific SAg-binding protein as used herein thus excludes polyclonal antibodies and antisera.

A SAg-binding protein of the invention, that can bind to, that has affinity for and/or that has specificity for a specific target molecule (antigenic determinant, epitope, antigen or protein) may be said to be “against” or “directed against” said target molecule. The term “specificity” refers to the number of different types of antigens or antigenic determinants to which a particular antigen-binding protein molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein. Alternatively, the affinity can also be expressed as the affinity constant (K_{A}), which is 1/K_{D}. Affinity can be determined in a manner known per se, depending on the specific combination of antigen-binding protein and antigen of interest. Avidity is herein understood to refer to the strength of binding of a target molecule with multiple binding sites by a larger complex of binding agents, i.e. the strength of binding of multivalent binding. Avidity is related to both the affinity between an antigenic determinant and its antigen-binding site on the antigen-binding molecule and the number of binding sites present on the antigen-binding molecule. Affinity, on the other hand refers to simple monovalent receptor ligand systems.

Typically, SAg-binding proteins of the invention will bind the target molecule with a dissociation constant (K_{D}) of about 10⁻⁵ to 10⁻¹² M or less, and preferably 10⁻⁷ to 10⁻¹² M or less and more preferably 10⁻⁸ to 10⁻¹² M or less, and/or with a binding affinity of at least 10⁻⁷ M, preferably at least 10⁻⁸ M, more preferably at least 10⁻⁹ M, such as at least 10^{-10, 10}⁻¹¹, 10⁻¹² M or more. Any K_{D} value greater than 10⁻⁴ M (i.e. less than 100 μM) is generally considered to indicate non-specific binding. Preferably, a polypeptide of the invention will bind to the desired antigen with an affinity less than 500 nM, preferably less than 200 nM, more preferably less than 10 nM, such as less than 500 pM. Specific binding of a SAg-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

Preferably, the SAg-binding protein comprising an amino acid sequence that comprises 4 framework regions, FR1 to FR4, and 3 complementarity determining regions, CDR1 to CDR3, that are operably linked in the order FR1-CDR1-FR2-CDR2-FR3-CDR3-FR4, wherein: a) the CDR1 has an amino acid sequence selected from the group consisting of SEQ ID No's: 72 to 138 or an amino acid sequence that differs from SEQ ID No's: 72 to 138 in one or two of the amino acid residues; b) the CDR2 has an amino acid sequence having at least 80, 85, 90, 95, 98% sequence identity with an amino acid sequence selected from the group consisting of SEQ ID No's: 139 to 205; and, c) CDR3 is an amino acid sequence having at least 80, 85, 90, 95, 98% sequence identity with an amino acid sequence selected from the group consisting of SEQ ID No's: 206 to 272; and, wherein each of the framework regions has at least 50, 55, 60, 65, 70, 75, 80, 85, 90, 95 or 100% amino acid identity with the framework amino acid sequence of any one of SEQ ID No's: 1-4.

An epitope is defined as the portion of the target molecule that is bound by the SAg-binding protein. In case the SAg-binding protein is an antibody, the epitope is the portion of a target molecule that triggers an immunological response upon immunisation of an individual vertebrate host with this molecule. Generally it is the site of the target molecule where binding to an antibody takes place. The epitope is preferably present naturally in the target molecule. Optionally the epitope(s) is/are a sequence that has been artificially included in the target molecule. Optionally a multitude of the same or different epitopes is included in the target molecule to facilitate its purification and detection.

A target molecule is herein defined as a molecule that is to be bound by a binding agent, preferably a SAg-binding protein of the invention. A target molecule may be a protein that requires to adsorbed, inactivated, removed, depleted, purified, detected or identified in or from a given context or environment. A preferred target molecule in the context of the present invention is a superantigen or SAg as herein further defined. It is herein understood that a SAg-binding protein that binds to a first type of target molecule and not to a second type of target molecule has a difference in dissociation constants for the first and second types of target molecules, respectively of at least a factor 100, 1000, 10,000 or 100,000. Preferably, a SAg-binding protein of the invention does not bind to endogenous mammalian (in particular human) proteins, except that a SAg-binding protein of the invention may bind to endogenous mammalian or human SAgs as may be produced by cells infected by Epstein-Barr virus rabies, cytomegalovirus, HIV or to proteins having SAg activity as expressed by a mammalian or human endogenous retroviruses (see e.g. US20070249808).

An antigen-binding protein of the invention may bind to a SAg produced by or derived from viruses, mycoplasma, and bacteria. A preferred SAg-binding protein of the invention binds to a SAg produced by or derived from a bacterium of a genus selected from the group consisting of Staphylococcus, Streptococcus, Mycoplasma and Yersinia. More preferably the SAg-binding protein binds to a SAg produced by or derived from a bacterium of a species selected from the group consisting of Staphylococcus aureus, Streptococcus pyogenes, Mycoplasma arthritidis and Yersinia enterocolitica. S. pyogenes is also known as group A streptococcus or GAS.

Staphylococcal SAgs to which the SAg-binding proteins of the invention may bind include e.g. the Staphylococcal enterotoxins (SEs) A, B, C1-3, D, E, H and toxic shock syndrome toxin-1 (TS ST-1). Streptococcal SAgs to which the SAg-binding proteins of the invention may bind include e.g. the pyrogenic exotoxins (SPEs) A, C, H; the mitogenic exotoxins SMEZ and SMEZ-2; and Streptococcal superantigen (SSA). SAg-binding proteins of the invention may also bind to SAgs from Mycoplasma arthritidis such as MAM (Ribeiro-Dias et al., 2003, Exp Cell Res. 286(2):345-54) and to SAgs from Yersinia enterocolitica (Stuart et al., 1995, Hum Immunol. 1995 August ;43(4):269-75).

Bacterial SAgs are globular proteins of 22-29 kDa that are resistant to proteases and heat denaturation. Comparison of the threedimensional structures of SAgs reveals a conserved two domain architecture (N- and C-terminal domains) and the presence of a long, solvent-accessible α-helix spanning the centre of the molecule. Phylogenetic analysis of Staphylococcal and Streptococcal SAgs indicates a 20%-90% sequence similarity and suggests that all SAgs have evolved from a common ancestral gene. Based on amino acid sequence alignment of Streptococcal and Staphylococcal SAgs it is possible to divide them into three subfamilies: (1) SEA, SED, SEE, SEH, and SEI; (2) SEB, SECT-3, SPEA1-3, SSA, and SEG and (3) SPEC, SPEJ, SPEG, SMEZ, and SMEZ-2. TSST-1 has about 28% amino acid identitity with other SEs and cannot be grouped with any of these subfamilies.

A first preferred embodiment of the invention concerns SAg-binding proteins that bind TSST-1. A SAg-binding protein that binds TSST-1 preferably is an antigen-binding protein having a structure as herein defined above wherein: a) the CDR1 has an amino acid sequence selected from the group consisting of SEQ ID No's: 72-93 and amino acid sequences that differs from SEQ ID No's: 72-93 in no more than 4, 3, 2 or 1 amino acid residues; b) the CDR2 has an amino acid sequence selected from the group consisting of SEQ ID No's: 139-160 and amino acid sequences that differs from SEQ ID No's: 139-160 in no more than 6, 5, 4, 3, 2, or 1 amino acid residues; and, c) the CDR3 has an amino acid sequence selected from the group consisting of SEQ ID No's: 206-227 and amino acid sequences that differs from SEQ ID No's: 206-227 in no more than 6, 5, 4, 3, 2, or 1 amino acid residues. More preferably the SAg-binding protein that binds TSST-1 has an amino acid sequence selected from the group consisting of SEQ ID No's 5-26.

A second preferred embodiment of the invention concerns SAg-binding proteins that bind one or more of SEA, SED, SEE, SEH, and SEI. More preferably the SAg-binding protein binds at least SEA. A SAg-binding protein that binds one or more of SEA, SED, SEE, SEH, and SEI, preferably is an antigen-binding protein having a structure as herein defined above wherein: a) the CDR1 has an amino acid sequence selected from the group consisting of SEQ ID No's: 94-109 and amino acid sequences that differs from SEQ ID No's: 94-109 in no more than 4, 3, 2 or 1 amino acid residues; b) the CDR2 has an amino acid sequence selected from the group consisting of SEQ ID No's: 161-176 and amino acid sequences that differs from SEQ ID No's: 161-176 in no more than 6, 5, 4, 3, 2, or 1 amino acid residues; and, c) the CDR3 has an amino acid sequence selected from the group consisting of SEQ ID No's: 228-243 and amino acid sequences that differs from SEQ ID No's: 228-243 in no more than 6, 5, 4, 3, 2, or 1 amino acid residues. More preferably the SAg-binding protein that binds one or more of SEA, SED, SEE, SEH, and SEI, has an amino acid sequence selected from the group consisting of SEQ ID No's 27-42.

A third preferred embodiment of the invention concerns SAg-binding proteins that bind one or more of SEB, SEC1-3, SPEA1-3, SSA, and SEG. More preferably the SAg-binding protein binds at least SEB. A SAg-binding protein that binds one or more of SEB, SEC1-3, SPEA1-3, SSA, and SEG, preferably is an antigen-binding protein having a structure as herein defined above wherein: a) the CDR1 has an amino acid sequence selected from the group consisting of SEQ ID No's: 110-138 and amino acid sequences that differs from SEQ ID No's: 110-138 in no more than 4, 3, 2 or 1 amino acid residues; b) the CDR2 has an amino acid sequence selected from the group consisting of SEQ ID No's: 177-205 and amino acid sequences that differs from SEQ ID No's: 177-205 in no more than 6, 5, 4, 3, 2, or 1 amino acid residues; and, c) the CDR3 has an amino acid sequence selected from the group consisting of SEQ ID No's: 244-272 and amino acid sequences that differs from SEQ ID No's: 244-272 in no more than 6, 5, 4, 3, 2, or 1 amino acid residues. More preferably the SAg-binding protein that binds one or more of SEB, SEC1-3, SPEA1-3, SSA, and SEG, has an amino acid sequence selected from the group consisting of SEQ ID No's: 43-71.

A fourth preferred embodiment of the invention concerns SAg-binding proteins that bind one or more of SPEC, SPEJ, SPEG, SMEZ, and SMEZ-2.

In a further embodiment the invention pertains to particular form of SAg-binding proteins of the invention: a multivalent antigen-binding protein or multivalent SAg-binding protein. The multivalent SAg-binding protein comprises the amino acid sequences of at least two SAg-binding proteins as defined herein above. The amino acid sequences of at least two SAg-binding proteins may be different from each or they may be identical, e.g. copies or repeats of one amino acid sequence. The amino acid sequences of the at least two SAg-binding proteins will usually be fused head-to tail, i.e. the C-terminus of the most N-terminal sequence fused to the N-terminus of the second sequence and so on. The amino acid sequences of at least two SAg-binding proteins may be fused directly linked or via a linker or spacer. Multivalent SAg-binding proteins of the invention may be produced by expression of a nucleotide sequence encoding the multivalent protein wherein two or more coding sequences of the SAg-binding proteins are operably linked together in the same reading frame. The skilled person will know how to operably fuse protein coding sequences.

In a further preferred embodiment, an antigen-binding protein of the invention is an inhibitor of a SAg. An inhibitor of a SAg is herein understood to mean a compound (i.e. an antigen-binding protein) capable of reducing the overstimulation of the immune system by SAgs, the T cell proliferation by SAgs and/or the T cell anergy and apoptosis by SAgs. Preferably, an antigen-binding protein is defined as an inhibitor of superantigen if in a tritium-thymidin incorporation assay on human PBMCs, after stimulation of the PBMCs with SAg the tritium activity incorporated in the PBMCs in counts per minute at a concentration of 25-70 nM, preferably of about 30-55 nM, more preferably of about 33-40 nM, more preferably at least about 34 nM and even more preferably 34 nM of antigen-binding protein results in less than 20%, 30%, 40%, 50%, 55%, 60%, 65%, 70%, 75%, 80%, 85% or 90% of the tritium activity in counts per minute in a control sample with SAg stimulation but without antigen-binding protein, and preferably results in the tritium activity in counts per minute as incorporated in PBMCs without SAg stimulation. The skilled person is aware that the concentration of superantigen to stimulate the PBMCs should be sufficient to induce proliferation, however the concentration should not be so high that inhibition of proliferation is no longer possible. The skilled person can easily determine a suitable concentration by stimulating PBMCs with different concentrations of a superantigen and subsequently determining the tritium activity incorporated in the PBMCs as is done in the tritium-thymidin incorporation assay. The concentration of superantigen whereby proliferation is about 90-99.9% of maximum proliferation, is preferably used in the tritium-thymidin incorporation assay described above. Specific concentrations of preferred superantigens to stimulate PBMCs are for example 11.4 nM of TSST-1, 1.8 nM of SEA or 7.1 nM of SEB.

Detailed information on how to perform a tritium-thymidin incorporation assay is provided in Visvanathan et al (2001, Infect Immun. 69(2):875-84), Goodell et al. (2007; BMC Immunology 8:21) and in Example 1.2. of the present specification. In a preferred embodiment the SAg is TSST-1 and the antigen-binding protein of the invention is an inhibitor of TSST-1. More preferably, an antigen-binding protein that is an inhibitor of a SAg has a structure as herein defined above wherein: a) the CDR1 has an amino acid sequence as presented in SEQ ID No: 72 or an amino acid sequence that differs from SEQ ID No: 72 in no more than 4, 3, 2 or 1 amino acid residues; b) the CDR2 has i) an amino acid sequence as presented in SEQ ID No: 139, ii) an amino acid sequence that differs from SEQ ID No: 139 in no more than 6, 5, 4, 3, 2, or 1 amino acid residues or iii) an amino acid sequence having at least 80%, 85%, 90%, 95%, or 97% sequence identity with an amino acid sequence as presented in SEQ ID No: 139; and, c) the CDR3 has i) an amino acid sequence as presented in SEQ ID No: 206, ii) an amino acid sequence that differs from SEQ ID No: 206 in no more than 6, 5, 4, 3, 2, or 1 amino acid residues or iii) an amino acid sequence having at least 80%, 85%, 90%, 95% or 97% sequence identity with an amino acid sequence as presented in SEQ ID No: 206. More preferably the SAg-binding protein that inhibits a SAg has an amino acid sequence which has at least about 45%, 50%, 55%, 60%, 65%, 70%, 75%, 80%, 85%, 90%, 95%, 97%, 98% or 99% sequence identity with SEQ ID No 5. More preferably, the SAg-binding protein that inhibits a SAg has 100% sequence identity with SEQ ID NO:5.

Most of the other SAg-binding proteins as described above are not inhibitors of SAg. Without wishing to be bound by any theory, the SAg-binding protein with the sequence as presented in SEQ ID No:5 comprises a cysteine residue in CDR3, which is capable of forming a cysteine bridge with a cysteine residue at the N-terminus of FR2 and may provide a conformation that contributes to the SAg inhibitory effect.

In a second aspect the invention relates to a SAg-binding protein as herein defined above for use as a medicament.

In a third aspect the invention relates to a SAg-binding protein as herein defined above for use in the treatment or prevention of a condition caused by the SAg. Alternatively in this aspect the invention relates to the use of the SAg-binding protein for the manufacture of a medicament for the treatment or prevention of a condition caused by the SAg. Preferably, the condition caused by the SAg that is to be treated or prevented is a condition selected from the group consisting of Toxic Shock Syndrome, Toxic shock-like syndrome, sepsis, Kawasaki disease, a skin disease as defined elsewhere herein, rheumatoid arthritis, (insulin-dependent) diabetes mellitus, scarlet fever, multiple sclerosis, human immunodeficiency virus (HIV) infection and food poisoning, of which in particular Staphylococcal food poisoning (SFP).

In a fourth aspect the invention relates to a method for treating (and/or preventing) a condition caused by a SAg wherein the method comprises the step of administering an effective amount of a SAg-binding protein as herein defined above to a subject in need thereof. Preferably the subject is suffering or at risk of suffering from a condition selected from the group consisting of Toxic Shock Syndrome, Toxic shock-like syndrome, sepsis, Kawasaki disease, a skin disease elsewhere herein, rheumatoid arthritis, (insulin-dependent) diabetes mellitus, scarlet fever, multiple sclerosis, human immunodeficiency virus (HIV) infection and food poisoning, of which in particular Staphylococcal food poisoning (SFP).

SAg-binding proteins of the invention that are used in the above methods of treatment and/or prevention preferably are SAg-binding proteins that inactivate the SAg by binding to the SAg. The ability of an SAg-binding protein to inactivate an SAg may be determined in an SAg-activity assay as described above.

In a preferred embodiment, the use of SAg-binding proteins of the invention in methods for treating (and/or preventing) a condition caused by a SAg is combined with conventional treatment of SAg-based diseases. Such conventional treatments are primarily limited to supportive therapy once symptoms have manifested and treatments include e.g. antibiotic use, administration of intravenous fluids, and medications to treat low blood pressure and shock.

Toxic shock syndrome (TSS) is a serious, life threatening disease resulting from an infection of a susceptible host by Staphylococci- or Streptococci-expressing SAgs in vivo. TSST-1 is the key virulence factor responsible for TSS, inducing most TSS symptoms in animals. TSST-1 is responsible for nearly all menstrual TSS cases and approx 60% of nonmenstrual Staphylococcal TSS. In the United States of America approximately 20,000 cases of TSS occur each year with a 20% mortality rate. The remainder of the cases can be attributed primarily to SEB production, and to a lesser extent SEC and SEA. Streptococcal TSS, also referred to as Toxic shock-like syndrome (TSLS), can be attributed mainly to SPEA. Thus, in a preferred embodiment the SAg-binding proteins are use to treat or prevent menstrual TSS, non-menstrual Staphylococcal TSS, both caused by TSST-1, non-menstrual Staphylococcal TSS caused by one or more of SEA, -B and -C; and/or Streptococcal TSS, preferably Streptococcal TSS caused by SPEA.

Staphylococcal food poisoning (SFP) is the leading cause of microbial food-borne illness worldwide. Food sources that are contaminated most often include foods that are high in protein, salt, and sugar. Abdominal pain, nausea, vomiting, and diarrhea are commonly seen within 2-6 h of ingestion of contaminated food; the absence of fever suggests that toxemia is at most, minimal. Little is known about how SAg structure relates with emetic and diarrhea activity. It has been suggested that the symptoms of food poisoning are a result of the high levels of cytokines released following SAg-induced T-cell proliferation.

A skin disease that can be treated with an SAg-binding protein of the present invention, preferably is a skin disease caused by or associated with the presence of micro-organisms that produce superantigens. Preferably, the micro-organism causing or associated with the skin disease is Staphylococcus aureus. Without wishing to be bound to any theory, the antigen-binding protein of the invention may be used in treatment of any skin disease wherein the subject suffering from the disease has lesions (e.g. from scratching), since micro-organisms (e.g. S. aureus) may colonise the lesions and secrete SAg and structural molecules within the cell wall into the lesions. SAg may lead to corticosteroid resistance and may also induce skin inflammation, thereby slowing down lesion healing. A skin disease is preferably selected from the group consisting of atopic dermatitis (AD), staphylococcal scalded skin syndrome, staphylococcal scarlatiniform eruption, psoriasis vulgaris, cutaneous T-cell lymphoma and acute juvenile pityriasis rubra pilaris, micro-organism-related eczema and guttate psoriasis. More preferably, the skin disease is atopic dermatitis and even more preferably severe atopic dermatitis. About 90% of patients with atopic dermatitis (AD) are colonised with S. aureus, whereas only 5-10% of healthy individuals carry this micro-organism (Lin et al. (2007) Clinic Rev Allerg Immunol 33:167-177). S. aureus worsens AD by secreting SAgs and structural molecules within the cell wall that induce skin inflammation. Therefore, S. aureus on the skin may contribute to persistent skin inflammation and disease severity.

In a preferred embodiment an antigen-binding protein for use in the treatment of a skin disease is an inhibitor of a SAg as defined above.

It is known in the art that micro-organisms may become resistant towards antimicrobial treatments, such as erythromycin, azithromycin, clarithromycin, cephalosporin cefuroxime axetil, cephalexin, trimethoprim-sulfamethoxazole (co-trimoxazole), clyndamycin and penicillinase-resistant penicillins such as dicloxacillin, oxacillin, and cloxacillin. In patients with MRSA, clyndamycin, fusidic acid or combination therapy with trimethoprim-sulfamethoxazole and intranasal mupirocin are used to eliminate MRSA infection, however these therapies should be used sparingly in order to prevent resistance of MRSA towards these treatments. In addition, Hauk et al. disclosed that SAg are capable to induce glucocorticoid insensitivity (2000, J. Allergy Clin. Immunol. 105:782-787).

An SAg-binding protein that is capable of inhibiting an SAg as is defined above may reduce corticosteroid resistance and skin inflammation induced by SAg. Therefore, in a preferred embodiment, the use of SAg-binding proteins of the invention in methods for treating a skin disease further comprises a conventional treatment of a skin disease, preferably including administration of corticosteroids, even more preferably including topical administration of corticosteroids.

In a fifth aspect the invention pertains to a composition comprising a SAg-binding protein as defined herein. The composition preferably comprises at least one pharmaceutically acceptable carrier in addition to the SAg-binding protein. Any suitable pharmaceutically acceptable carrier or excipient can be used in pharmaceutical compositions with the SAg-binding proteins of the invention (See e.g., Remington: The Science and Practice of Pharmacy, Alfonso R. Gennaro (Editor) Mack Publishing Company, April 1997). Depending on the condition caused by a SAg to be treated (or prevented) a suitable mode of administration and corresponding pharmaceutical formulation can be selected.

For the treatment of Staphylococcal and Streptococcal TSS the preferred route of administration of the SAg-binding proteins of the invention is parenteral injection or infusion, i.e. preferably intravenous or intra-arterial. Compositions for parenteral administration will commonly comprise a solution of a SAg-binding protein of the invention dissolved in an acceptable carrier, preferably an aqueous carrier. A variety of aqueous carriers may be employed, e.g., water, buffered water, e.g. 0.3% glycine, 0.9% NaCl or 5% glucose, optionally supplemented with a 20% albumin solution. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the SAg-binding protein of the invention in such pharmaceutical formulation can vary widely, i.e., from less than about 0.5%, usually at or at least about 1% to as much as 15 or 20% by weight, and will be selected primarily based on fluid volumes, viscosities, etc., according to the particular mode of administration selected. Thus, a pharmaceutical composition of the invention for intramuscular injection could be prepared to contain 1 ml sterile buffered water, and 50 mg of a SAg-binding protein of the invention. Similarly, a pharmaceutical composition of the invention for intravenous infusion could be made up to contain 250 ml. of sterile Ringer's solution, and 150 mg of a SAg-binding protein of the invention. Actual methods for preparing parenterally administrable compositions are well known or will be apparent to those skilled in the art, and are described in more detail in, for example, Remington's Pharmaceutical Science, 15th ed., Mack Publishing Company, Easton, Pa., hereby incorporated by reference herein.

For the treatment of food poisoning, in particular SFP, the preferred route of admininistration of the SAg-binding proteins of the invention is by oral or enteral administration. For this purpose the SAg-binding protein of the invention can be administered in solid dosage forms, such as capsules, tablets, and powders, or in liquid dosage forms, such as elixirs, syrups, and suspensions. SAg-binding protein(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

For the treatment of a skin disease as defined elsewhere herein (such as atopic dermatitis), the preferred route of administration of the SAg-binding proteins of the invention is topical. Compositions for topical administration will commonly comprise a solution of a SAg-binding protein of the invention together with one or more acceptable carrier(s) therefor and optionally any other therapeutic ingredients(s). The carrier(s) must be “acceptable” in the sense of being compatible with the other ingredients of the formulation and not deleterious to the recipient thereof. Formulations suitable for topical administration include liquid or semi-liquid preparations suitable for penetration through the skin to the site of where treatment is required, such as liniments, lotions, creams, ointments or pastes, and drops suitable for administration to the eye, ear or nose. In relation to topical administration, it is known that conventional antibodies, i.e. an antibody comprising both heavy and light chains, is not suitable for topical administration, probably due to instability. Therefore, conventional antibodies are preferably excluded from the antigen-binding proteins of the invention that inhibit SAg. In contrast, antibodies or fragments thereof are derived from antibodies naturally devoid of light chains are more stable (Dolk et al. (2005) Applied and Environmental Microbiology 71:442-450) and therefore preferred for topical administration.

Drops according to the present invention may comprise sterile aqueous or oily solutions or suspensions and may be prepared by dissolving the SAg-binding protein in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 90°-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the SAg-binding protein for external application. They may be made by mixing the SAg-binding protein in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Specific examples of the various formulation with the SAg-binding proteins of the invention are given in the Examples herein.

In a sixth aspect the invention pertains to a composition a SAg-binding protein as defined herein immobilised onto a carrier. A preferred embodiment thereof is an immunoadsorbent material comprising the SAg-binding protein. An immunoadsorbent material is herein understood to mean the combination of a carrier and a SAg-binding protein that is immobilized on the carrier. Preferably in the immunoadsorbent material the SAg-binding protein is immobilized onto a carrier, whereby more preferably, the SAg-binding protein is immobilised onto the carrier by a covalent link. The carrier may be any material that may be used for immobilization of an antigen-binding protein. Suitable examples are matrix materials, to entrap the antigen-binding protein, cell surfaces on which the antigen-binding protein is displayed and polymers that can be covalently linked to the antigen-binding protein. The person skilled in the art of affinity chromatography is well aware of suitable carriers such as e.g. porous solid phase carrier materials such as agarose, polystyrene, controlled pore glass, cellulose, dextrans, kieselguhr, synthetic polymers such as SepharoseTM, porous amorphous silica. The carrier materials may be in any suitable format such as particles, powders, sheets, beads, filters and the like. Further specifications of suitable carrier materials are for example disclosed in EP-A-434317. Methods are available for immobilizing antigen-binding protein quickly, easily and safely through a chosen functional group. The correct choice of coupling method depends on the substance to immobilised. For example the following commercially known derivatives of Sepharose™ allow the convenient immobilisation of proteins thereon: CNBr-activated Sepharose™ 4B enables antigen-binding proteins containing primary amino groups to be rapidly immobilised by a spontaneous reaction. AH-Sepharose™ 4B and CH-Sepharose™ 4B both have a six-carbon long spacer arm and permit coupling via carboxyl and amino groups respectively. Flexible spacers are suitable for use in situations where the flexibility of the target molecules is limited or where 3-dimensional structure of the target requires some flexibility of the antigen-binding protein to allow optimal binding. NHS-activated Sepharose™ 4B Fast Flow provides a six-carbon spacer arm and an active ester for spontaneous coupling via amino groups. For non-covalent immobilisation SAg-binding proteins may produced with a His-tag that allows binding of the protein to metal affinity resins as described in El Khattabi et al. (2006, Clin. Vaccine Immunol. 13: 1079-1086). These are only a few examples of suitable immobilisation routes. Optionally the immunoadsorbent material is put into a column to facilitate easy chromatographic separations.

In a seventh aspect the invention relates to an immunoadsorbent material comprising a SAg-binding protein as herein defined above for use in the treatment or prevention of a condition caused by the SAg. Alternatively in this aspect the invention relates to the use of the immunoadsorbent material comprising the SAg-binding protein for the manufacture of a medicament for the treatment or prevention of a condition caused by the SAg. Preferably, the condition caused by the SAg that is to be treated or prevented is a condition selected from the group consisting of Toxic Shock Syndrome, sepsis, Kawasaki disease, atopic dermatitis (AD), eczema, guttate psoriasis, rheumatoid arthritis, (insulin-dependent) diabetes mellitus, scarlet fever, multiple sclerosis, human immunodeficiency virus (HIV) infection and food poisoning, of which in particular Staphylococcal food poisoning (SFP).

In an eighth aspect the invention relates to a method for treating (or preventing) a condition caused by a SAg wherein the method comprises the step of administering an effective amount of an immunoadsorbent material comprising a SAg-binding protein as herein defined above to a subject in need thereof. Preferably the subject is suffering or at risk of suffering from a condition selected from the group consisting of Toxic Shock Syndrome, Toxic shock-like syndrome, sepsis, Kawasaki disease, atopic dermatitis (AD), eczema, guttate psoriasis, rheumatoid arthritis, (insulin-dependent) diabetes mellitus, scarlet fever, multiple sclerosis, human immunodeficiency virus (HIV) infection and food poisoning, of which in particular Staphylococcal food poisoning (SFP).

In a ninth aspect the invention pertains to a method for adsorbing or scavenging a superantigen from a fluid. The method preferably is an in vitro method. The fluid may be any fluid containing, suspected to contain a superantigen, or that is required to be free of superantigen. Such fluids may e.g. include a body fluid, such as blood or a fraction thereof such as plasma, but also e.g. a feed stream obtained from (bacterial) fermentations (e.g. E. coli). The method preferably comprises the step (a) of bringing an immunoadsorbent material comprising a SAg-binding protein as herein defined above into contact with the fluid. The method may further comprise the step (b) of (physically) separating the fluid from the immunoadsorbent material comprising the adsorbed superantigen. To ensure complete depletion of the superantigen from the fluid, fluid obtained from step (b) may be subjected to a new step (a) with fresh immunoadsorbent material. The method may optionally comprise the step of recovery of the fluid depleted from superantigen.

In a preferred embodiment the invention relates to methods for therapeutic apheresis. Therapeutic apheresis is an extracorporeal blood treatment to eliminate pathogenic compounds from the blood (Bosch, 2003, J. Artif. Organs 6(1): 1-8). One example of therapeutic apheresis concerns the adsorption of in a variety of conditions caused by superantigens as defined above. Preferably the method for therapeutic apheresis comprises at least one of removing, depleting and inactivating a superantigen in or from a body fluid. Preferably the removing, depleting and inactivating of the superantigen in or from a body fluid is performed ex vivo. The method for therapeutic apheresis of the invention is thus an in vitro method. The body fluid preferably is blood, a blood fraction such as e.g. blood plasma or blood serum, or another body fluid. In the method an antigen-binding protein of the invention as defined hereinabove or an immunoadsorbent material comprising the antigen-binding protein as defined above, is brought into extracorporeal contact with the body fluid of a subject, preferably a human subject. The immunoadsorbent apheresis material may be in the form of particles, beads, filters or membranes, which may advantageously be packed into a flow chamber or a column, through which the body fluid of the subject or patient is passed extracorporeally. Before or after a treatment in which superantigen is depleted, one or more further treatment stages for the body fluid can be carried out. Several treatments of the body fluid can be carried out in successive units, in which superantigen is depleted by adsorption, to achieve the desired end concentration of superantigen. Samples of the body fluid before and after superantigen depletion may be tested using e.g. ELISA for superantigen levels (using e.g. the antigen-binding proteins of the invention). The body fluid may then be reinfused into the subject or human patient, although the latter step may be explicitly excluded from a preferred extracorporal embodiment of the method. In preferred embodiments the methods of the invention for therapeutic apheresis are applied on body fluids from patient or subjects suffering from a condition caused by a superantigen. Preferably, the condition caused by the SAg that is to be treated or prevented is a condition selected from the group consisting of Toxic Shock Syndrome, Toxic shock-like syndrome, sepsis, Kawasaki disease, atopic dermatitis (AD), eczema, guttate psoriasis, rheumatoid arthritis, (insulin-dependent) diabetes mellitus, scarlet fever, multiple sclerosis, human immunodeficiency virus (HIV) infection and food poisoning, of which in particular Staphylococcal food poisoning (SFP).

In a further preferred embodiment of the invention the methods for therapeutic apheresis are combined with conventional treatment of of SAg-based diseases. Such conventional treatments are primarily limited to supportive therapy once symptoms have manifested and treatments include e.g. antibiotic use, administration of intravenous fluids, and medications to treat low blood pressure and shock. The methods for therapeutic apheresis of the invention may also be combined with the use of the non-immobilised SAg-binding proteins of the invention as described above in methods for treating (and/or preventing) conditions caused by a SAg, optionally further in combination with the conventional treatments.

In a tenth aspect the invention thus also pertains to the use of a SAg-binding protein of the invention for extracorporeal adsorption, removal and/or depletion of a superantigen in a subject's body fluid, preferably a human subject.

In an eleventh aspect the invention relates to an apheresis device comprising the immunoadsorbent material of the invention comprising the SAg-binding protein as defined above. The apheresis device will usually be a container comprising the SAg-binding immunoadsorbent material of the invention. Preferably the apheresis device is a cartridge for integration into an apheresis system. Typically, the apheresis device will be constructed as a cylinder with an inlet to allow plasma to enter at one end, and an outlet at the opposite end to allow the cleaned plasma to exit and be returned to the patient. Other device configurations may also be designed and are within the scope of this invention. The cartridge device is constructed of material that is nontoxic and which provides rigid support to the carrier material (e.g. agarose) of the immunoadsorbent material within. Typically, the material will be a plastic composition such as polystyrene, or polyvinyl, or polypropylene or polycarbonate or other similar material. There is an inside filter at the bottom of the device to prevent the carrier (e.g. agarose) beads from leaving the device. There is also an inside filter at the top of the device to contain the carrier (e.g. agarose) within the device. Typically these filters are composed of plastic and/or cellulosic material and have pores that will allow thru passage of fluid such as plasma, but not particulate material such as agarose beads. The manufacture of these types of devices and the materials used are known to those skilled in the art and are within the scope of this invention.

In an twelfth aspect the invention the invention relates to an apheresis system comprising the SAg-binding immunoadsorbent material of the invention. An immunoadsorbent material of the invention comprising the SAg-binding protein as defined above may be integrated into any apheresis system substituting the immunoadsorbent material of the invention, as described above, for the corresponding part(s) associated with binding/removal of the target agent. The resulting SAg apheresis system then can be employed for SAg adsorption, removal depletion in accordance with the invention. Existent and already known apheresis devices in all of their embodiments can easily be adapted to the present invention. In particular, when choosing the solid carrier (and the apheresis device), the medical-technical usefulness thereof should be considered. Such carriers, methods or devices have been described i.a. in WO 97/48483 A, in U.S. Pat. Nos. 5,476,715, 6,036,614, 5,817,528 or 6,551,266. Corresponding commercial apheresis apparatus are marketed by the companies Fresenius, Affina, Plasmaselect, ASAHI, Kaneka, Braun, etc., such as, e.g., the LDL-Therasorb™, the Immunosorba™, the Prosorba™, the Globaffin™, the Ig-Therasorb™, the Immusorba™, the Liposorba™, the HELP™, the DALI™, the bilirubin-bile acid absorber BR-350, the Prometheus™ detoxication, the MARS™, the ADAsorb-System from Medicap or the Plasma FLO-System. All these systems, even though in their commercial form not primarily always aimed at the specific elimination of a single protein such as a superantigen, may be adapted to the present invention without any problems by a person skilled in apheresis, e.g. as immunapheresis and/or by installing the inventive solid carrier (e.g. as a column) in the apheresis device.

For example, the SAg-binding immunoadsorbent material of the invention could be substituted in apparatus with a design along the lines of the Adacolumn™, a single-use adsorptive apheresis device, which is connected to a blood pump with flow rate detector, pressure monitor and air sensor, or the Adacircuit™ infusion line system (G. Wehrli, 2005, Current Hematology reports, 4:477-482.). In this design, two 16-18 gauge needles are inserted into vascular access sites, such as bilateral antecubital fossa veins. The element is primed with saline optionally containing heparin. The priming fluid is collected in a waste container after the procedure begins. The patient's whole blood is continuously drawn into the line circuit using a blood pump and heparin is added into the line blood prior to entering the device. Treated whole blood then is returned to the patient, via the contralateral vascular access, and no replacement fluid is required. A typical procedure likely would take about sixty minutes.

In another aspect the invention relates to a nucleic acid comprising a nucleotide sequence encoding a SAg-binding protein as defined herein above. A preferred nucleic acid according to the invention is a nucleic acid construct, wherein the nucleotide sequence encoding the SAg-binding protein is operably linked to a promoter and optionally other regulatory elements such as e.g. terminators, enhancers, polyadenylation signals, signal sequences for secretion and the like. Such nucleic acid constructs are particularly useful for the production of the SAg-binding proteins of the invention using recombinant techniques in which a nucleotide sequence encoding the SAg-binding protein of interest is expressed in suitable host cells such as described in Ausubel et al., “Current Protocols in Molecular Biology”, Greene Publishing and Wiley-Interscience, New York (1987) and in Sambrook and Russell (2001) “Molecular Cloning: A Laboratory Manual (3^{rd} edition), Cold Spring Harbor Laboratory, Cold Spring Harbor Laboratory Press, New York). As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

In a further aspect the invention pertains to a host cell comprising a nucleic acid as defined above. Preferably the host cell is a host cell for production of SAg-binding protein of the invention. The host cell may be any host cell capable of producing a SAg-binding protein of the invention, including e.g. a prokaryotic host cell, such as e.g., E. coli, or a (cultured) mammalian, plant, insect, fungal or yeast host cell, including e.g. CHO-cells, BHK-cells, human cell cell lines (including HeLa, COS and PER.C6), Sf9 cells and Sf+ cells. A preferred host cell for production of a SAg-binding protein of the invention is however a cell of an eukaryotic microorganism such as yeasts and filamentous fungi. Preferred yeast host cell e.g. include e.g. Saccharomyces cerevisiae, Pichia pastoris, Hansenula polymorphs, and Kluyveromyces lactis. Preferred strains, constructs and fermentation conditions for production of the SAg-binding protein of the invention are described by van de Laar, et al., (2007, Biotechnology and Bioengineering, Vol. 96, No. 3: 483-494). For example, production of the antigen-binding proteins can be performed in standard bioreactors with a working volume between 10 and 10,000 litres. Dissolved oxygen can be controlled by automatic adjustment of the impeller speed. The pH can be controlled using phosphoric acid and ammoniac gas or ammonia solution and temperature controlled via e.g. a cooling jacket and heating jacket. The offgas is analysed on ethanol concentration, rO₂ and rCO₂. The batch phase is started by adding 3% -8% of full-grown inoculum (e.g. 30° C., 0.3-0.4 VVM air, DO₂ minimum 30%, pH 5.0). When the ethanol concentration in offgas is declining in batch phase the ethanol fermentation can be started. The feed can be applied according to a pulsed feed profile to maintain the ethanol level within the demanded margins. The feed phases can be performed at 21° C. and 0.7-1.1 VVM air. During the ethanol fermentations DO₂ decreases to 0% and accumulated ethanol can be further controlled by a pulsed feed profile. Feed phase stops when the ethanol feed is depleted. The broth can be chilled to a temperature between 5-10° C. till further processing like biomass removal etc. (VVM=volumes of air per minute per volume of batch). In this context it is also understood that whenever herein we a SAg-binding protein of the invention as being obtainable by expression in yeast at a certain minimal expression level, this level is obtained using the method as described in Example 1.1. herein, whereby the (maximal) concentration of the SAg-binding protein (at the end of fermentation) “g/L” refers to the amount of secreted SAg-binding protein (in grams) per liter of cell-free broth (i.e., after removal of biomass by e.g filtration). A preferred SAg-binding protein of the invention is obtainable by expression in yeast at an expression level of at least 0.5, 0.8, 1.0, 1.2, 1.5, 2.0 or 2.5 g/L of yeast culture.

Thus, in yet another aspect the invention relates to a method for producing a SAg-binding protein of the invention, wherein the method preferably comprises the steps of: a) culturing a host cell as defined above under conditions conducive to expression of the SAg-binding protein; and optionally, b) purifying the SAg-binding protein from at least one of the host cell and the culture medium. Suitable conditions may include the use of a suitable medium, the presence of a suitable source of food and/or suitable nutrients, a suitable temperature, and optionally the presence of a suitable inducing factor or compound (e.g. when the nucleotide sequences of the invention are under the control of an inducible promoter); all of which may be selected by the skilled person. Under such conditions, the amino acid sequences of the invention may be expressed in a constitutive manner, in a transient manner, or only when suitably induced. The SAg-binding proteins of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the SAg-binding protein to be isolated).

In a yet a further aspect the invention relates to a method of detecting a SAg in a sample. The method prefereably comprises the steps of a) contacting a sample with an SAg-binding protein as defined above; b) detecting binding of the SAg-binding protein to the sample, or determining the amount of SAg in the sample. The method preferably is an in vitro method. In one embodiment the method is a method of diagnosing a condition caused by a SAg as defined above. The diagnostic method optionally comprises the further step c) of comparing the binding detected in step (b) with a standard, wherein a difference in binding relative to said sample is diagnostic of the condition caused by a SAg, or comparing the amount determined in step (b) with a standard, wherein a difference in amount relative to said sample is diagnostic of the condition caused by a SAg.

In the diagnostic method of the invention, a sample is obtained, or collected, from a subject to be tested for a condition caused by a SAg. The subject may or may not be suspected of having a such condition. A sample is any specimen obtained from the subject that can be used to measure the amount of SAg. A preferred sample is a bodily fluid that can be used to measure the amount of SAg. Those skilled in the art can readily identify appropriate samples.

As used herein, the term “contacting” refers to the introduction of a sample putatively containing a SAg to an SAg-binding protein, e.g., by combining or mixing the sample with the respective polypeptide(s). When SAg is present in the sample, a complex is then formed; such complex can be detected. Detection can be qualitative, quantitative, or semi-quantitative.

Binding SAg in the sample to the respective SAg-binding protein is accomplished under conditions suitable to form a complex. Such conditions (e.g. appropriate concentrations, buffers, temperatures, reaction times) as well as methods to optimize such conditions are known to those skilled in the art. Binding can be measured using a variety of methods standard in the art including, but not limited to, enzyme immunoassays (e. g., ELISA), immunoprecipitations, immunoblot assays and other immunoassays as described, for example, in Sambrook et al., supra, and Harlow et al., Antibodies, a Laboratory Manual (Cold Spring Harbor Labs Press, 1988). These references also provide examples of complex formation conditions. In one embodiment, the aforementioned complex can be formed in solution. In another embodiment, the aforementioned complex can be formed in which one component (either SAg or the SAg-binding protein) is immobilized on (e.g., coated onto) a substrate. Immobilization techniques are known to those skilled in the art. Suitable substrate materials include, but are not limited to, plastic, glass, gel, celluloid, fabric, paper, and particulate materials. Examples of substrate materials include, but are not limited to, latex, polystyrene, nylon, nitrocellulose, agarose, cotton, PVDF (poly-vinylidene-fluoride), and magnetic resin. Suitable shapes for substrate material include, but are not limited to, a well (e.g., microtiter dish well), a microliter plate, a dipstick, a strip, a bead, a lateral flow apparatus, a membrane, a filter, a tube, a dish, a celluloid-type matrix, a magnetic particle, and other particulates. Particularly preferred substrates include, for example, an ELISA plate, a dipstick, an immunodot strip, a radioimmunoassay plate, an agarose bead, a plastic bead, a latex bead, a sponge, a cotton thread, a plastic chip, an immunoblot membrane, an immunoblot paper and a flow-through membrane. In one embodiment, a substrate, such as a particulate, can include a detectable marker. For descriptions of examples of substrate materials, see, for example, Kemeny, D. M. (1991) A Practical Guide to ELISA, Pergamon Press, Elmsford, N.Y. pp 33-44, and Price, C. and Newman, D. eds. Principles and Practice of Immunoassay, 2nd edition (1997) Stockton Press, NY, N.Y., both of which are incorporated herein by reference in their entirety. In a preferred embodiment, an SAg-binding protein is immobilized on a substrate, such as a microtiter dish well, a dipstick, an immunodot strip, or a lateral flow apparatus. A sample collected from a subject is applied to the substrate and incubated under conditions suitable (i.e., sufficient) to allow for complex formation bound to the substrate.

In accordance with the present invention, once formed, a complex is detected. As used herein, the term “detecting complex formation” refers to identifying the presence of SAg-binding protein complexed to SAg. If complexes are formed, the amount of complexes formed can, but need not be, quantified. Complex formation, or selective binding, can be measured (i.e., detected, determined) using a variety of methods standard in the art (see, for example, Sambrook et al. supra.), examples of which are disclosed herein. A complex can be detected in a variety of ways including, but not limited to use of one or more of the following assays: an enzyme-linked immunoassay, a competitive enzyme-linked immunoassay, a radioimmunoassay, a fluorescence immunoassay, a chemiluminescent assay, a lateral flow assay, a flow-through assay, an agglutination assay, a particulate-based assay (e.g., using particulates such as, but not limited to, magnetic particles or plastic polymers, such as latex or polystyrene beads), an immunoprecipitation assay, a BioCore assay (e.g., using colloidal gold), an immunodot assay (e.g., CMG's Immunodot System, Fribourg, Switzerland), and an immunoblot assay (e.g., a western blot), an phosphorescence assay, a flow-through assay, a particulate-based assay, a chromatography assay, a PAGE-based assay, a surface plasmon resonance assay, a spectrophotometric assay and an electronic sensory assay. Such assays are well known to those skilled in the art.

Assays can be used to give qualitative or quantitative results depending on how they are used. The assay results can be based on detecting the entire SAg molecule or fragments, degradation products or reaction products thereof. Some assays, such as agglutination, particulate separation, and immunoprecipitation, can be observed visually (e.g., either by eye or by a machines, such as a densitometer or spectrophotometer) without the need for a detectable marker.

In other assays, conjugation of a detectable marker to the SAg-binding protein or its target SAg aids in detecting complex formation. For example, a detectable marker can be conjugated to the SAg-binding protein at a site that does not interfere with their ability to bind their respective targets. Methods of conjugation are known to those skilled in the art. Examples of detectable markers include, but are not limited to, a radioactive label, a fluorescent label, a chemiluminescent label, a chromophoric label, an enzyme label, a phosphorescent label, an electronic label; a metal sol label, a colored bead, a physical label, or a ligand. A ligand refers to a molecule that binds selectively to another molecule. Preferred detectable markers include, but are not limited to, fluorescein, a radioisotope, a phosphatase (e.g., alkaline phosphatase), biotin, avidin, a peroxidase (e.g., horseradish peroxidase), beta-galactosidase, and biotin-related compounds or avidin-related compounds (e.g., streptavidin or ImmunoPure NeutrAvidin).

The present invention can further comprise one or more layers and/or types of secondary molecules or other binding molecules capable of detecting the presence of an indicator molecule. For example, an untagged (i.e., not conjugated to a detectable marker) secondary antibody that selectively binds to an SAg-binding protein can be bound to a tagged tertiary antibody that selectively binds to the secondary antibody. Suitable secondary antibodies, tertiary antibodies and other secondary or tertiary molecules can be readily selected by those skilled in the art. Preferred tertiary molecules can also be selected by those skilled in the art based upon the characteristics of the secondary molecule. The same strategy can be applied for subsequent layers.

Depending on the assay, a developing agent is added and the substrate is submitted to a detection device for analysis. In some protocols, washing steps are added after one or both complex formation steps in order to remove excess reagents. If such steps are used, they involve conditions known to those skilled in the art such that excess reagents are removed but the complex is retained. Once the level of SAg has been measured, an assessment of whether a condition caused by an SAg is present can then be made. Assessing the presence of such condition means comparing the level of SAg in the test sample to the level found in healthy subjects. The presence of SAg in the sample is indicative of such condition.

A diagnostic kit according to the invention comprises all the necessary means and media for performing the detection of SAg or fragment thereof by interaction an SAg-binding protein. The kit is useful for diagnosis of conditions caused by SAg. According to one aspect of the invention, a diagnostic kit comprises one or more SAg-binding proteins as described herein.

According to another aspect of the invention, a diagnostic kit comprises one or more recombinant cells of the invention, comprising and expressing the nucleotide sequence encoding an SAg-binding protein. Kits useful according to the invention can comprise an isolated SAg-binding protein, a homologue thereof, or a functional portion thereof. A kit according to the invention can comprise cells transformed to express said polypeptide. Kits useful according to the invention can include an isolated SAg or fragment thereof. Alternatively, or in addition, a kit can comprise cells transformed to express SAg or fragment thereof. In a further embodiment, a kit according to the invention can comprise a polynucleotide encoding an SAg or fragment thereof. In a still further embodiment, a kit according to the invention may comprise the specific primers useful for amplification of an SAg or fragment thereof. All kits according to the invention will comprise the stated items or combinations of items and packaging materials therefore. Kits will also include instructions for use. SAg, fragments thereof, and/or SAg-binding protein may be supplied immobilised, e.g., on a microtitre plate, on a glass chip suitable for high-throughput screening, on magnetic beads, or on an insoluble solid support.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

TABLE 1
Non-limiting examples of amino acid residues
in FR1. Indicated in bold is the consensus
sequence for the SAg-binding sequences. The
other a.a. occur at these positions also in
camelid antibodies.

| | a.a.residue anti-SAg | a.a.residue |
|---|---|---|
| Position | a.a. sequences | Camelid VHH's |
| 1 | Q | A E |
| 2 | V | |
| 3 | Q | K |
| 4 | L | |
| 5 | Q | E L V |
| 6 | E D | Q A |
| 7 | S | F |
| 8 | G | |
| 9 | G | |
| 10 | G A | D R |
| 11 | L S | M V W |
| 12 | V | A |
| 13 | Q R | E K P |
| 14 | A P T | Q A G S V |
| 15 | G | |
| 16 | G D E A | |
| 17 | S | F |
| 18 | L | V |
| 19 | R S T N | K L |
| 20 | L I | V F |
| 21 | S | A F T |
| 22 | C | |
| 23 | A V E T K L | R S D P |
| 24 | A V F | P D I L S T |
| 25 | S K | L A F P T |
| 26 | G E | Q P A D R S T V |

TABLE 2
Non-limiting examples of amino acid residues in
FR2. Indicated in bold is the consensus
sequence for the SAg-binding sequences. The
other a.a. occur at these positions also
in camelid antibodies.

| | a.a.residue anti-SAg | a.a.residue |
|---|---|---|
| Position | a.a. sequences | Camelid VHH's |
| 36 | W | |
| 37 | F Y L | H I V |
| 38 | R H | |
| 39 | Q | R E H P |
| 40 | A T P V G | F L |
| 41 | P S | L A |
| 42 | G | E |
| 43 | K N | T A D E Q R V |
| 44 | E Q D L K M | G A R S |
| 45 | R | L C I L P Q V |
| 46 | E D Q | K V |
| 47 | F L G W I | S A M R V Y |
| 48 | V L I | |
| 49 | A S G | T V |

TABLE 3
Non-limiting examples of amino acid residues
in FR3. Indicated in bold is the consensus
sequence for the SAg-binding sequences. The
other a.a. occur at these
positions also in camelid antibodies.

| | a.a.residue anti-SAg | a.a.residue |
|---|---|---|
| Position | a.a. sequences | Camelid VHH's |
| 65 | G A | D |
| 66 | R | |
| 67 | F Y | L V |
| 68 | T A | N S |
| 69 | I V M | T L |
| 70 | S Y T | F A |
| 71 | R K S | G N M H I L Q T W |
| 72 | D E | G N V |
| 73 | N S A D I T V H | R K Y G F L |
| 74 | A T V G | P D N S |
| 75 | K Q G E R M | N H A L |
| 76 | N K S | D Y T R |
| 77 | T M A S V | E I P |
| 78 | V M A G L N | I E F |
| 79 | Y D S T R | F H N F A |
| 80 | L | F V |
| 81 | Q E R D H | V L I T |
| 82 | M | L I V |
| 82a | N S D I | T G H |
| 82b | S G R N V F | D T |
| 82c | L V | I P |
| 83 | K Q E R | N G I M T |
| 84 | P A L S T | V F D R |
| 85 | E D | G Q |
| 86 | D | |
| 87 | T S | A |
| 88 | A G | S |
| 89 | V I L | M S R A D M N T |
| 90 | Y N | F |
| 91 | Y S H R N | F L D T V |
| 92 | C | |
| 93 | A N T | H V Y S F G K R |
| 94 | A T V | L S I G R C F K |

TABLE 4
Non-limiting examples of amino acid residues
in FR4. Indicated in bold is the consensus
sequence for the SAg-binding sequences.
The other a.a. occur at these
positions also in camelid antibodies.

| | a.a.residue | a.a.residue |
|---|---|---|
| Position | anti-SAg a.a. sequences | Camelid VHH's |
| 103 | W G | P R S |
| 104 | G | R A D |
| 105 | Q K P | E R |
| 106 | G | |
| 107 | T I | A |
| 108 | Q L H | P R |
| 109 | V I | |
| 110 | T A N S | I |
| 111 | V | A I |
| 112 | S | F |
| 113 | S A | L P T |

### EXAMPLES

### Example 1.1

### Production of Superantigen-Binding VHH Fragments

The SAg-binding proteins of the invention were produced in yeast using strains and expression-constructs as described by van de Laar, et al., (2007, Biotechnology and Bioengineering, Vol. 96, No. 3: 483-494). Production of SAg-binding proteins was performed in standard bioreactors with a working volume of between 10 and 10,000 litres. Dissolved oxygen (Ingold DO2 electrode, Mettler-Toledo) was controlled by automatic adjustment of the impeller speed. The pH (Mettler-Toledo Inpro 3100 gel electrode or Broadley James F635 gel electrode) was controlled using phosphoric acid and ammoniac gas or ammonia solution. Foaming was detected by a foam level sensor (Thermo Russell) and controlled by 5-10% Struktol J673 addition. Temperature (PT100 electrode) was controlled via a cooling jacket and heating jacket. The offgas (Prima 600 mass spectrophotometer, VG gas analysis systems) analysed the ethanol concentration, rO2 and rCO2. Adding 3% -8% full-grown inoculum started the batch phase (30° C., 0.3-0.4 VVM air, DO2 minimum 30%, pH 5.0). The ethanol fermentations were automatically started when the ethanol concentration in offgas was declining in batch phase. The feed was applied according to a pulsed feed profile to maintain the ethanol level within the demanded margins. The feed phases were performed at 21° C. and 0.7-1.1 VVM air. During the ethanol fermentations the DO2 decreased to 0% and accumulated ethanol was further controlled by a pulsed feed profile. Feed phase was stopped when the ethanol feed was depleted. The broth was chilled to a temperature between 5-10° C. until further processing, including removal of spent biomass removal.

Typical fermentation parameters include a temperature of 20-31° C., a pH of 4.7-5.8, product (SAg-binding protein) formed: 1000-1500 mg/l cell free broth, fermentation time of 115-120 h and cell dry weight (at the end of fermentation): 95-115 g/kg.

### Example 1.2

### Specific Inhibition of TSST-1 Induced Cell Proliferation by Anti-TSST-1 VHH

For a typical inhibition experiment adapted from Visvanathan et al. (2001, Infect Immun. 69(2):875-84), human peripheral blood mononuclear cells (PBMCs) were isolated by standard Ficoll-Hypaque techniques: using Ficoll-Paque Plus according to the manufacturer's instructions (GE Healthcare, Sweden) and adjusted to 2×10⁶ cells/ml. PBMCs (2×10⁵) in 200 μl of complete medium (RPMI medium 1640+10% human type AB serum; Sigma-Aldrich, St. Louis, Mo.) were placed in 96-well titer plates (Nunc, Roskilde, Denmark) and stimulated with various doses of superantigen (250 ng/ml [=11.4 nM] TSST-1, 50 ng/ml [=1.8 nM] Enteroxin A or 200 ng/ml [=7.1 nM] Enterotoxin B; all from Sigma-Aldrich) or with a combination of each toxin and various doses of anti-TSST-1 VHH (Table 5). The cells were incubated for 4 days, and proliferation of the cells was measured via tritiated thymidine incorporation as described in Goodell et al. (2007; BMC Immunology 8:21). Briefly, the method is as follows: after incubation of the cells for 4 days at 37° C. in an incubator (5% CO₂), 1 μCi ³H-Thymidin solution per well was added. The cells were incubated for another 24 h at 37° C. Cells were harvested and supernatant was transferred into another plate. 50 μl Trypsin/EDTA was added to the cells, followed by incubation until all cells were detached. The supernatants were pipetted back into the corresponding wells containing the detached cells, and cells were harvested on filter discs. Scintillation fluid was added and radioactivity was counted in a β-counter (Beckmann Coulter, CA, USA).

The data presented are averages of the results of three different experiments. The tests were done in duplo. As shown in Table 5, a significant reduction of radioactive thymidin incorporation can be reached when more than 500 ng/ml (=34 nM) anti-TSST-1 is applied in the experiment. The inhibition of TSST-1 proliferation by anti-TSST-1 VHH is specific as the reduction is not observed when Enterotoxin A or Enterotoxin B is used as proliferation-inducing agent.

TABLE 5
Decreased TSST-1 induced proliferation of PBMCs by anti-TSST-1 VHH.

| anti-TSST-1 | | | | | | | EntA | EntB | TSST-1 |
|---|---|---|---|---|---|---|---|---|---|
| VHH | [³H]-Thymidin incorporation (cpm) | | | | | | activity | activity | activity |
| ng/ml | AVG | SD | AVG | SD | AVG | SD | % | % | % |
| 0 | 229388 | 4827 | 225885 | 2630 | 176979 | 10821 | 100 | 100 | 100 |
| 10 | 249700 | 7607 | 245518 | 3441 | 187434 | 10592 | 109 | 109 | 106 |
| 50 | 242118 | 3039 | 240708 | 7540 | 172475 | 3651 | 106 | 107 | 97 |
| 100 | 235466 | 29145 | 273038 | 6366 | 181613 | 6919 | 103 | 121 | 103 |
| 500 | 286881 | 7241 | 245697 | 8608 | 118767 | 11376 | 125 | 109 | 67 |
| 1000 | 274407 | 21591 | 230081 | 1935 | 84492 | 2116 | 120 | 102 | 48 |

Abbreviations: AVG: average, SD, standard deviation.

### Example 2

### Formulations with the SAg-Binding Proteins of the Invention

### 2.1 Capsule Composition

A pharmaceutical composition of this invention in the form of a capsule is prepared by filling a standard two-piece hard gelatin capsule with 50 mg of a SAg-binding protein of the invention, in powdered form, 100 mg of lactose, 32 mg of talc and 8 mg of magnesium stearate.

### 2.2 Ointment Composition

SAg-binding protein of the invention 1.0 g; White soft paraffin to 100.0 g. The SAg-binding protein of the invention is dispersed in a small volume of the vehicle to produce a smooth, homogeneous product. Collapsible metal tubes are then filled with the dispersion.

### 2.3 Topical Cream Composition

SAg-binding protein of the invention 1.0 g; Polawax GP 200 20.0 g; Lanolin Anhydrous 2.0 g; White Beeswax 2.5 g; Methyl hydroxybenzoate 0.1 g; Distilled Water to 100.0 g. The polawax, beeswax and lanolin are heated together at 60° C. A solution of methyl hydroxybenzoate is added and homogenization is achieved using high speed stirring. The temperature is then allowed to fall to 50° C. The SAg-binding protein of the invention is then added and dispersed throughout, and the composition is allowed to cool with slow speed stirring.

### 2.4 Topical Lotion Composition

SAg-binding protein of the invention 1.0 g; Sorbitan Monolaurate 0.6 g; Polysorbate 20 0.6 g; Cetostearyl Alcohol 1.2 g; Glycerin 6.0 g; Methyl Hydroxybenzoate 0.2 g; Purified Water B.P. to 100 ml. (B.P.=British Pharmacopeia). The methyl hydroxybenzoate and glycerin are dissolved in 70 ml. of the water at 75° C. The sorbitan monolaurate, polysorbate 20 and cetostearyl alcohol are melted together at 75° C. and added to the aqueous solution. The resulting emulsion is homogenized, allowed to cool with continuous stirring and the SAg-binding protein of the invention is added as a suspension in the remaining water. The whole suspension is stirred until homogenized.

### 2.5 Eye Drop Composition

SAg-binding protein of the invention 0.5 g; Methyl Hydroxybenzoate 0.01 g; Propyl Hydroxybenzoate 0.04 g; Purified Water B.P. to 100 ml. The methyl and propyl hydroxybenzoates are dissolved in 70 ml. purified water at 75° C. and the resulting solution is allowed to cool. The SAg-binding protein of the invention is then added, and the solution is sterilized by filtration through a membrane filter (0.022 μm pore size), and packed aseptically into suitable sterile containers.

1. An antigen-binding protein for use in the treatment of a skin disease, wherein the antigen-binding protein is an inhibitor of a superantigen.

2. A method of treating a skin disease in a subject in need thereof comprising administering the antigen-binding protein of claim 1 in an amount effective to inhibit a superantigen.

3. An antigen-binding protein according to claim 1, wherein the antigen-binding protein is defined as an inhibitor of superantigen if in a tritium-thymidine incorporation assay on human PBMCs, after stimulation of the PBMCs with superantigen, the tritium activity incorporated in the PBMCs in counts per minute at a concentration of at least 34 nM of antigen-binding protein is less than 70% of the tritium activity in counts per minute in a control sample without antigen-binding protein, and preferably results in the tritium activity in counts per minute as incorporated in PBMCs without superantigen stimulation.

4. An antigen-binding protein according to claim 3, wherein the PBMCs are stimulated with 11.4 nM TSST-1, 1.8 nM SEA or 7.1 nM SEB.

5. An antigen-binding protein according to claim 1, wherein the superantigen is TSST-1.

6. An antigen-binding protein according to claim 1, wherein the antigen-binding protein comprises an immunoglobulin-derived variable domain that comprises a complete antigen binding site for an epitope on TSST-1 in a single polypeptide chain.

7. The method according to claim 2, wherein the skin disease is a micro-organism related skin disease, selected from the group consisting of atopic dermatitis, staphylococcal scalded skin syndrome, staphylococcal scarlatiniform eruption, guttate psoriasis, psoriasis vulgaris, cutaneous T-cell lymphoma, micro-organism-related eczema and acute juvenile pityriasis rubra pilaris.

8. An antigen-binding protein according to claim 1, wherein the antigen-binding protein comprises an amino acid sequence that comprises 4 framework regions, FR1 to FR4, and 3 complementarity determining regions, CDR1 to CDR3, that are operably linked in the order FR1-CDR1-FR2-CDR2-FR3-CDR3-FR4, wherein:
(a) the CDR1 has an amino acid sequence as presented in SEQ ID NO: 72 or an amino acid sequence that differs from SEQ ID NO: 72 in one or two of the amino acid residues;
(b) the CDR2 has an amino acid sequence having at least 80% sequence identity with an amino acid sequence as presented in SEQ ID NO: 139 and,
(c) the CDR3 is an amino acid sequence having at least 80% sequence identity with an amino acid sequence as presented in SEQ ID NO: 206 and,
wherein each of the framework regions has at least 50% amino acid identity with the framework amino acid sequence of any one of SEQ ID NO: 1-4.

9. An antigen-binding protein or a use according to claim 8, wherein the antigen-binding protein comprises an amino acid sequence which has at least 60% amino acid identity with the amino acid sequence as presented in SEQ ID NO: 5.

10. The method according to claim 2, wherein the treatment further comprises a conventional treatment of a skin disease, with corticosteroids.

11. The method according to claim 2, wherein the antigen-binding protein is administered topically in a formulation selected from the group consisting of liniments, lotions, creams, ointments or pastes, and drops suitable for administration to the eye, ear or nose.

12. An antigen-binding protein comprising an amino acid sequence that comprises 4 framework regions, FR1 to FR4, and 3 complementarity determining regions, CDR1 to CDR3, that are operably linked in the order FR1-CDR1-FR2-CDR2-FR3-CDR3-FR4, wherein:
(a) the CDR1 has an amino acid sequence as presented in SEQ ID NO: 72 or an amino acid sequence that differs from SEQ ID NO: 72 in one or two of the amino acid residues;
(b) the CDR2 has an amino acid sequence having at least 80% sequence identity with an amino acid sequence as presented in SEQ ID NO: 139; and,
(c) the CDR3 is an amino acid sequence having at least 80% sequence identity with an amino acid sequence as presented in SEQ ID NO: 206; and,
wherein each of the framework regions has at least 50% amino acid identity with the framework amino acid sequence of any one of SEQ ID NO: 1-4.

13. An antigen-binding protein according to claim 12, wherein the antigen-binding protein comprises an amino acid sequence which has at least 60% amino acid identity with the amino acid sequence as presented in SEQ ID NO: 5

14. (canceled)

15. A composition comprising an antigen-binding protein as defined in claim 12 and a pharmaceutically acceptable carrier, wherein the composition is formulated for topical administration.
